(19) United States
(12) Patent Application Publication
(10) Pub. No.: US 2016/0130594 A1
(43) Pub. Date: May 12, 2016

(54) SYNTHETIC BI-DIRECTIONAL PLANT PROMOTER

(71) Applicant: Dow AgroSciences LLC, Indianapolis, IN (US)

(72) Inventors: Sandeep Kumar, Carmel, IN (US); Jeffrey R. Berringer, Carmel, IN (US); Wei Chen, Carmel, IN (US); Andrew M. Asberry, Indianapolis, IN (US)

(21) Appl. No.: 14/938,632

(22) Filed: Nov. 11, 2015

Related U.S. Application Data
(60) Provisional application No. 62/078,214, filed on Nov. 11, 2014.

(51) Int. Cl. C12N 15/82

(52) U.S. Cl. CPC C12N 15/8216; C12N 15/8205; C12N 15/8207; C12N 15/8286; C12N 15/8274; C12N 15/8261; C12N 15/8206

(57) ABSTRACT

This disclosure concerns compositions and methods for promoting transcription of a nucleotide sequence in a plant or plant cell, employing a minimal core promoter element from a Zea mays Ubiquitin-1 gene promoter, and the full-length nucleotide sequence elements from a Rice Ubiquitin-3 promoter. Some embodiments relate to a synthetic bi-directional promoter that may function in plants to promote transcription of two operably linked nucleotide sequences.

### CROSS-REFERENCE TO RELATED APPLICATION

This application claims the benefit of the filing date of U.S. Provisional Patent Application Ser. No. 62/078,214, filed Nov. 11, 2014, for “SYNTHETIC BI-DIRECTIONAL PLANT PROMOTER,” the disclosure of which is hereby incorporated herein in its entirety by this reference.

### TECHNICAL FIELD

The present disclosure generally relates to compositions and methods for promoting transcription of a nucleotide sequence in a plant or plant cell. Some embodiments relate to a synthetic Rice Ubiquitin-3 (Rubi3) bi-directional promoter that functions in plants to promote transcription of an operably linked nucleotide sequence. Particular embodiments relate to methods including a synthetic promoter (e.g., to introduce a nucleic acid molecule into a cell) and cells, cell cultures, tissues, organisms, and parts of organisms comprising a synthetic promoter, as well as products produced therefrom.

### BACKGROUND

Many plant species are capable of being transformed with transgenes from other species to introduce agronomically desirable traits or characteristics, for example, improving nutritional value quality, increasing yield, conferring pest or disease resistance, increasing drought and stress tolerance, improving horticultural qualities (such as pigmentation and growth), imparting herbicide resistance, enabling the production of industrially useful compounds and/or materials from the plant, and/or enabling the production of pharmaceuticals. The introduction of transgenes into plant cells and the subsequent recovery of fertile transgenic plants that contain a stably integrated copy of the transgene can result in the production of transgenic plants that possess the desirable traits or characteristics.

Control and regulation of gene expression can occur through numerous mechanisms. Transcription initiation of a gene is a predominant controlling mechanism of gene expression. Initiation of transcription is generally controlled by polynucleotide sequences located in the 5′-flanking or upstream region of the transcribed gene. These sequences are collectively referred to as promoters. Promoters generally contain signals for RNA polymerase to begin transcription so that messenger RNA (mRNA) can be produced. Mature mRNA is transcribed by ribosomes, thereby synthesizing proteins. DNA-binding proteins interact specifically with promoter DNA sequences to promote the formation of a transcriptional complex and initiate the gene expression process. There are a variety of eukaryotic promoters isolated and characterized from plants that are functional for driving the expression of a transgene in plants. Promoters that affect gene expression in response to environmental stimuli, nutrient availability, or adverse conditions including heat shock, anaerobiosis, or the presence of heavy metals have been isolated and characterized. There are also promoters that control gene expression during development or in a tissue, or organ specific fashion. In addition, prokaryotic promoters isolated from bacteria and viruses have been isolated and characterized that are functional for driving the expression of a transgene in plants.

A typical promoter that is capable of expression in a eukaryote consists of a minimal promoter and other cis-elements. The minimal promoter is essentially a TATA box region where RNA polymerase II (polII), TATA-binding protein (TBP), and TBP-associated factors (TAFs) may bind to initiate transcription. However, in most instances, sequence elements other than the TATA motif are required for accurate transcription. Such sequence elements (e.g., enhancers) have been found to elevate the overall level of expression of the nearby genes, often in a position- and/or orientation-independent manner. Other sequences near the transcription start site (e.g., INR sequences) of some polII genes may provide an alternate binding site for factors that also contribute to transcriptional activation, even alternatively providing the core promoter binding sites for transcription in promoters that lack functional TATA elements. Zenzie-Gregory et al. (1992) J. Biol. Chem. 267: 2823-30.

Other gene regulatory elements include sequences that interact with specific DNA-binding factors. These sequence motifs are sometimes referred to as cis-elements, and are usually position- and orientation-dependent, though they may be found 5′ or 3′ to a gene's coding sequence, or in an intron. Such cis-elements, to which tissue-specific or development-specific transcription factors bind, individually or in combination, may determine the spatiotemporal expression pattern of a promoter at the transcriptional level. The arrangement of upstream cis-elements, followed by a minimal promoter, typically establishes the polarity of a particular promoter. Promoters in plants that have been cloned and widely used for both basic research and biotechnological application are generally unidirectional, directing only one gene that has been fused at its 3′ end (i.e., downstream). See, Xie et al. (2001) Nat. Biotechnol. 19(7):677-9; U.S. Pat. No. 6,388,170.

Many cis-elements (or “upstream regulatory sequences”) have been identified in plant promoters. These cis-elements vary widely in the type of control they exert on operably linked genes. Some elements act to increase the transcription of operably-linked genes in response to environmental responses (e.g., temperature, moisture, and wounding). Other cis-elements may respond to developmental cues (e.g., germination, seed maturation, and flowering) or to spatial information (e.g., tissue specificity). See, e.g., Langridge et al. (1989) Proc. Natl. Acad. Sci. USA 86:3219-23. The type of control of specific promoter elements is typically an intrinsic quality of the promoter; i.e., a heterologous gene under the control of such a promoter is likely to be expressed according to the control of the native gene from which the promoter element was isolated. Id. These elements also typically may be exchanged with other elements and maintain their characteristic intrinsic control over gene expression.

It is often necessary to introduce multiple genes into plants for metabolic engineering and trait stacking, which genes are frequently controlled by identical or homologous promoters. However, homology-based gene silencing (HBGS) is likely to arise when multiple introduced transgenes have homologous promoters driving them. Mol et al. (1989) Plant Mol. Biol. 13:287-94. Thus, HBGS has been reported to occur extensively in transgenic plants. See, e.g., Vaucheret and Fagard (2001) Trends Genet. 17:29-35. Several mechanisms have been suggested to explain the phenomena of HBGS, all of which include the feature that sequence homology in the promoter triggers cellular recognition mechanisms that result in silencing of the repeated genes. Matzke and Matzke (1995) 47:23-48; Fire (1999) Trends Genet. 15:358-63; Hamilton and Baulcombe (1999) Science 286:950-2; Steimer et al. (2000) Plant Cell 12:1165-78. Furthermore, the repeated use of the same promoter to obtain similar levels of expression patterns of different transgenes can result in an excess of competing transcription factor (TF)-binding sites in repeated promoters can cause depletion of endogenous TFs and lead to transcriptional downregulation.

Given that there is an ever greater need for integration of robustly expressing multigenic traits within a single locus of a transgenic event; solutions that provide for reducing the technical challenges associated with creating such transgenic events are of importance. More specifically, strategies to avoid HBGS in transgenic plants that involve the development of synthetic promoters that are functionally equivalent but have minimal sequence homology are desirable. When such synthetic promoters are used for expressing transgenes in crop plants, they may aid in avoiding or reducing HBGS. Mourrain et al. (2007) Planta 225(2):365-79; Bhullar et al. (2003) Plant Physiol. 132:988-98.

### BRIEF SUMMARY

In embodiments of the subject disclosure, the disclosure relates to a synthetic Rice Ubiquitin-3 bi-directional polynucleotide promoter comprising a plurality of promoter elements from a Rice Ubiquitin-3 promoter and a Zea mays Ubiquitin-1 promoter. In a further embodiment, the subject disclosure comprises various promoter elements. Accordingly, the promoter elements comprise an intron. In some instances the promoter elements comprise a 5′-UTR. In addition, the promoter elements comprise an upstream promoter element. Furthermore, the promoter elements comprise a minimal core promoter. In embodiments of the subject disclosure, the disclosure relates to a method for producing a transgenic plant cell, comprising the steps of: a) transforming a plant cell with a gene expression cassette comprising a synthetic Rice Ubiquitin-3 bi-directional polynucleotide promoter operably linked to at least one polynucleotide sequence of interest; b) isolating the transformed plant cell comprising the gene expression cassette; and, c) producing a transgenic plant cell comprising the synthetic Rice Ubiquitin-3 bi-directional polynucleotide promoter operably linked to at least one polynucleotide sequence of interest. In embodiments of the subject disclosure, the disclosure relates to a method for expressing a polynucleotide sequence of interest in a plant cell, the method comprising introducing into the plant cell the polynucleotide sequence of interest operably linked to a synthetic Rice Ubiquitin-3 bi-directional polynucleotide promoter. In embodiments of the subject disclosure, the disclosure relates to a transgenic plant cell comprising the synthetic Rice Ubiquitin-3 bi-directional polynucleotide promoter.

The foregoing and other features will become more apparent from the following detailed description of several embodiments, which proceeds with reference to the accompanying figures.

### BRIEF DESCRIPTION OF THE FIGURES

FIG. 1: Plasmid map of pDAB113122.

FIG. 2: Plasmid map of pDAB113142.

FIG. 3: A graph of the Cry34 expression in V6 leaf tissue of corn plants transformed with either construct pDAB113122 or pDAB113142.

FIG. 4: A graph of Cry35 expression in V6 leaf tissue of corn plants transformed with either construct pDAB113122 or pDAB113142.

FIG. 5: A graph of AAD-1 expression in V6 leaf tissue of corn plants transformed with either construct pDAB113122 or pDAB113142.

### DETAILED DESCRIPTION

### I. Overview of Several Embodiments

Development of transgenic plants is becoming increasingly complex, and typically requires stacking multiple transgenes into a single locus. See Xie et al. (2001) Nat. Biotechnol. 19(7):677-9. Since each transgene usually requires a unique promoter for expression, multiple promoters are required to express different transgenes within one gene stack. In addition to increasing the size of the gene stack, this frequently leads to repeated use of the same promoter to obtain similar levels of expression patterns of different transgenes. This approach is often problematic, as the expression of multiple transgenes driven by the same promoter may lead to gene silencing or HBGS. An excess of competing transcription factor (TF)-binding sites in repeated promoters can cause depletion of endogenous TFs and lead to transcriptional downregulation. The silencing of transgenes is undesirable to the performance of a transgenic plant produced to express the transgenes. Repetitive sequences within a transgene often lead to intra-locus homologous recombination resulting in polynucleotide rearrangements and undesirable phenotypes or agronomic performance.

Plant promoters used for basic research or biotechnological application are generally unidirectional, and regulate only one gene that has been fused at its 3′ end (downstream). To produce transgenic plants with various desired traits or characteristics, it would be useful to reduce the number of promoters that are deployed to drive expression of the transgenes that encode the desired traits and characteristics. Especially in applications where it is necessary to introduce multiple transgenes into plants for metabolic engineering and trait stacking, thereby necessitating multiple promoters to drive the expression of multiple transgenes. By developing a single Rice Ubiquitin-3 synthetic bi-directional promoter that can drive expression of two transgenes that flank the promoter, the total numbers of promoters needed for the development of transgenic crops may be reduced, thereby lessening the repeated use of the same promoter, reducing the size of transgenic constructs, and/or reducing the possibility of HBGS. Such a promoter can be generated by introducing known cis-elements in a novel or synthetic stretch of DNA, or alternatively by “domain swapping,” wherein domains of one promoter are replaced with functionally equivalent domains from other heterologous promoters.

Embodiments herein utilize a process wherein a unidirectional promoter from a Oryza sativa (Rice) Ubiquitin-3 gene (e.g., Rubi3) was used to design a synthetic Rice Ubiquitin-3 bi-directional promoter, such that one promoter can direct the expression of two genes, one on each end of the promoter. Synthetic Rice Ubiquitin-3 bi-directional promoters may allow those in the art to stack transgenes in plant cells and plants while lessening the repeated use of the same promoter and reducing the size of transgenic constructs. Furthermore, regulating the expression of two genes with a single synthetic Rice Ubiquitin-3 bi-directional promoter may also provide the ability to co-express the two genes under the same conditions, such as may be useful, for example, when the two genes each contribute to a single trait in the host. The use of bi-directional function of promoters in plants has been reported in some cases, including the Zea mays Ubiquitin 1 promoter (International Patent Publication No. WO2013101343 A1), CaMV 35 promoters (Barfield and Pua (1991) Plant Cell Rep. 10(6-7):308-14; Xie et al. (2001), supra), and the mas promoters (Velten et al. (1984) EMBO J. 3(12):2723-30; Langridge et al. (1989) Proc. Natl. Acad. Sci. USA 86:3219-23).

Transcription initiation and modulation of gene expression in plant genes is directed by a variety of DNA sequence elements that are collectively arranged within the promoter. Eukaryotic promoters consist of minimal core promoter element (minP), and further upstream regulatory sequences (URSs). The core promoter element is a minimal stretch of contiguous DNA sequence that is sufficient to direct accurate initiation of transcription. Core promoters in plants also comprise canonical regions associated with the initiation of transcription, such as CAAT and TATA boxes. The TATA box element is usually located approximately 20 to 35 nucleotides upstream of the initiation site of transcription.

The activation of the minP is dependent upon the URS, to which various proteins bind and subsequently interact with the transcription initiation complex. URSs comprise of DNA sequences, which determine the spatiotemporal expression pattern of a promoter comprising the URS. The polarity of a promoter is often determined by the orientation of the minP, while the URS is bipolar (i.e., it functions independent of its orientation).

In specific examples of some embodiments, a minimal core promoter element (minUbi10P) of a modified Zea mays Ubiquitin-1 promoter (ZmUbi1) originally derived from Zea mays, is used to engineer a synthetic Rice Ubiquitin-3 bi-directional promoter that functions in plants to provide expression control characteristics that are unique with respect to previously described bi-directional promoters. Embodiments include a synthetic Rice Ubiquitin-3 bi-directional promoter that further includes a minimal core promoter element nucleotide sequence derived from a native Zea mays Ubiquitin-1 promoter (minPZmUbi1).

The ZmUbi1 promoter originally derived from Zea mays c.v. B73 comprises sequences located in the maize genome within about 899 bases 5′ of the transcription start site, and further within about 1,093 bases 3′ of the transcription start site. Christensen et al. (1992) Plant Mol. Biol. 18(4):675-89 (describing a Zea mays c.v. B73 ZmUbi1 gene). A modified ZmUbi1 promoter derived from B73 that is used in some examples is an approximately 2 kb promoter that contains a TATA box; two overlapping heat shock consensus elements; an 82 or 83 nucleotide (depending on the reference strand) leader sequence immediately adjacent to the transcription start site, which is referred to herein as ZmUbi1 exon; and a 1015-1016 nucleotide intron. Other maize ubiquitin promoter variants derived from Zea species and Zea mays genotypes may exhibit high sequence conservation around the minP element consisting of the TATA element and the upstream heat shock consensus elements. Thus, embodiments of the invention are exemplified by the use of this short (˜200 nt) highly-conserved region (e.g., SEQ ID NO:2) of a ZmUbi1 promoter as a minimal core promoter element for constructing synthetic bidirectional plant promoters.

The Rice Ubiquitin-3 promoter originally derived from Oryza sativa comprises sequences located in the rice genome within about 1,990 bases 5′ of the transcription start site. E Sivamani, and R Qu (2006) Expression enhancement of a rice polyubiquitin promoter. Plant Molecular Biology 60: 225-239. A modified Rice Ubiquitin-3 promoter derived from Oryza sativa that is used in some examples is an approximately 2 kb promoter that contains a TATA box, a 5′ UTR/intron sequence, and a downstream enhancing element located at the start of the Rice Ubiquitin-3 coding sequence. Other Rice Ubiquitin-3 promoter variants derived from Oryza species and Oryza sativa genotypes may exhibit high sequence conservation around these promoter elements.

### II. Abbreviations

AtUbi10 Arabidopsis thaliana Ubiquitin 10

BCA bicinchoninic acid

CaMV cauliflower mosaic virus

CsVMV cassava vein mosaic virus

CTP chloroplast transit peptide

HBGS homology-based gene silencing

minUbi1P ZmUbi1 minimal core promoter

OLA oligo ligation amplification

PCR polymerase chain reaction

RCA rolling circle amplification

RUbi3 Rice Ubiquitin-3

RT-PCR reverse transcriptase PCR

SNuPE single nucleotide primer extension

URS upstream regulatory sequence

ZmUbi1 Zea Mays Ubiquitin 1

### III. Terms

Throughout the application, a number of terms are used. In order to provide a clear and consistent understanding of the specification and claims, including the scope to be given such terms, the following definitions are provided:

Introns: As used herein, the term “intron” refers to any nucleic acid sequence comprised in a gene (or expressed polynucleotide sequence of interest) that is transcribed but not translated. Introns include untranslated nucleic acid sequence within an expressed sequence of DNA, as well as the corresponding sequence in RNA molecules transcribed therefrom.

Isolated: An “isolated” biological component (such as a nucleic acid or protein) has been substantially separated, produced apart from, or purified away from other biological components in the cell of the organism in which the component naturally occurs (i.e., other chromosomal and extra-chromosomal DNA and RNA, and proteins), while effecting a chemical or functional change in the component (e.g., a nucleic acid may be isolated from a chromosome by breaking chemical bonds connecting the nucleic acid to the remaining DNA in the chromosome). Nucleic acid molecules and proteins that have been “isolated” include nucleic acid molecules and proteins purified by standard purification methods. The term also embraces nucleic acids and proteins prepared by recombinant expression in a host cell, as well as chemically-synthesized nucleic acid molecules, proteins, and peptides.

Gene expression: The process by which the coded information of a nucleic acid transcriptional unit (including, e.g., genomic DNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

Homology-based gene silencing: As used herein, “homology-based gene silencing” (HBGS) is a generic term that includes both transcriptional gene silencing and post-transcriptional gene silencing. Silencing of a target locus by an unlinked silencing locus can result from transcription inhibition (transcriptional gene silencing; TGS) or mRNA degradation (post-transcriptional gene silencing; PTGS), owing to the production of double-stranded RNA (dsRNA) corresponding to promoter or transcribed sequences, respectively. The involvement of distinct cellular components in each process suggests that dsRNA-induced TGS and PTGS likely result from the diversification of an ancient common mechanism. However, a strict comparison of TGS and PTGS has been difficult to achieve because it generally relies on the analysis of distinct silencing loci. We describe a single transgene locus that triggers both TGS and PTGS, owing to the production of dsRNA corresponding to promoter and transcribed sequences of different target genes. Mourrain et al. (2007) Planta 225:365-79. It is likely that siRNAs are the actual molecules that trigger TGS and PTGS on homologous sequences: the siRNAs would in this model trigger silencing and methylation of homologous sequences in cis and in trans through the spreading of methylation of transgene sequences into the endogenous promoter. Id.

Nucleic acid molecule: As used herein, the term “nucleic acid molecule” (or “nucleic acid” or “polynucleotide”) may refer to a polymeric form of nucleotides, which may include both sense and anti-sense strands of RNA, cDNA, genomic DNA, and synthetic forms and mixed polymers of the above. A nucleotide may refer to a ribonucleotide, deoxyribonucleotide, or a modified form of either type of nucleotide. A “nucleic acid molecule,” as used herein, is synonymous with “nucleic acid” and “polynucleotide.” A nucleic acid molecule is usually at least 10 bases in length, unless otherwise specified. The term may refer to a molecule of RNA or DNA of indeterminate length. The term includes single- and double-stranded forms of DNA. A nucleic acid molecule may include either or both naturally-occurring and modified nucleotides linked together by naturally occurring and/or non-naturally occurring nucleotide linkages.

Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications (e.g., uncharged linkages: for example, methyl phosphonates, phosphotriesters, phosphoramidates, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages: for example, alpha anomeric nucleic acids, etc.). The term “nucleic acid molecule” also includes any topological conformation, including single-stranded, double-stranded, partially duplexed, triplexed, hairpinned, circular, and padlocked conformations.

Transcription proceeds in a 5′ to 3′ manner along a DNA strand. This means that RNA is made by the sequential addition of ribonucleotide-5′-triphosphates to the 3′ terminus of the growing chain (with a requisite elimination of the pyrophosphate). In either a linear or circular nucleic acid molecule, discrete elements (e.g., particular nucleotide sequences) may be referred to as being “upstream” or “5′” relative to a further element if they are bonded or would be bonded to the same nucleic acid in the 5′ direction from that element. Similarly, discrete elements may be “downstream” or “3′” relative to a further element if they are or would be bonded to the same nucleic acid in the 3′ direction from that element.

A base “position,” as used herein, refers to the location of a given base or nucleotide residue within a designated nucleic acid. The designated nucleic acid may be defined by alignment (see below) with a reference nucleic acid.

Hybridization: Oligonucleotides and their analogs hybridize by hydrogen bonding, which includes Watson-Crick, Hoogsteen or reversed Hoogsteen hydrogen bonding, between complementary bases. Generally, nucleic acid molecules consist of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between two distinct nucleic acid sequences or two distinct regions of the same nucleic acid sequence.

“Specifically hybridizable” and “specifically complementary” are terms that indicate a sufficient degree of complementarity such that stable and specific binding occurs between the oligonucleotide and the DNA or RNA target. The oligonucleotide need not be 100% complementary to its target sequence to be specifically hybridizable. An oligonucleotide is specifically hybridizable when binding of the oligonucleotide to the target DNA or RNA molecule interferes with the normal function of the target DNA or RNA, and there is sufficient degree of complementarity to avoid non-specific binding of the oligonucleotide to non-target sequences under conditions where specific binding is desired, for example, under physiological conditions in the case of in vivo assays or systems. Such binding is referred to as specific hybridization.

Hybridization conditions resulting in particular degrees of stringency will vary depending upon the nature of the chosen hybridization method and the composition and length of the hybridizing nucleic acid sequences. Generally, the temperature of hybridization and the ionic strength (especially the Na+ and/or Mg2+ concentration) of the hybridization buffer will contribute to the stringency of hybridization, though wash times also influence stringency. Calculations regarding hybridization conditions required for attaining particular degrees of stringency are discussed in Sambrook et al. (ed.), Molecular Cloning: A Laboratory Manual, 2nd ed., vol. 1-3, Cold Spring Harbor Laboratory Press, Cold Spring Harbor, N. Y., 1989, chs. 9 and 11.

As used herein, “stringent conditions” encompass conditions under which hybridization will only occur if there is less than 50% mismatch between the hybridization molecule and the DNA target. “Stringent conditions” include further particular levels of stringency. Thus, as used herein, “moderate stringency” conditions are those under which molecules with more than 50% sequence mismatch will not hybridize; conditions of “high stringency” are those under which sequences with more than 20% mismatch will not hybridize; and conditions of “very high stringency” are those under which sequences with more than 10% mismatch will not hybridize.

In particular embodiments, stringent conditions can include hybridization at 65° C., followed by washes at 65° C. with 0.1×SSC/0.1% SDS for 40 minutes.

The following are representative, non-limiting hybridization conditions:
Very High Stringency: Hybridization in 5×SSC buffer at 65° C. for 16 hours; wash twice in 2×SSC buffer at room temperature for 15 minutes each; and wash twice in 0.5×SSC buffer at 65° C. for 20 minutes each.
High Stringency: Hybridization in 5×-6×SSC buffer at 65-70° C. for 16-20 hours; wash twice in 2×SSC buffer at room temperature for 5-20 minutes each; and wash twice in 1×SSC buffer at 55-70° C. for 30 minutes each.
Moderate Stringency: Hybridization in 6×SSC buffer at room temperature to 55° C. for 16-20 hours; wash at least twice in 2×-3×SSC buffer at room temperature to 55° C. for 20-30 minutes each.

In particular embodiments, specifically hybridizable nucleic acid molecules can remain bound under very high stringency hybridization conditions. In these and further embodiments, specifically hybridizable nucleic acid molecules can remain bound under high stringency hybridization conditions. In these and further embodiments, specifically hybridizable nucleic acid molecules can remain bound under moderate stringency hybridization conditions.

Oligonucleotide: An oligonucleotide is a short nucleic acid polymer. Oligonucleotides may be formed by cleavage of longer nucleic acid segments, or by polymerizing individual nucleotide precursors. Automated synthesizers allow the synthesis of oligonucleotides up to several hundred base pairs in length. Because oligonucleotides may bind to a complementary nucleotide sequence, they may be used as probes for detecting DNA or RNA. Oligonucleotides composed of DNA (oligodeoxyribonucleotides) may be used in PCR, a technique for the amplification of small DNA sequences. In PCR, the oligonucleotide is typically referred to as a “primer,” which allows a DNA polymerase to extend the oligonucleotide and replicate the complementary strand.

Sequence identity: The term “sequence identity” or “identity,” as used herein, in the context of two nucleic acid or polypeptide sequences, may refer to the residues in the two sequences that are the same when aligned for maximum correspondence over a specified comparison window.

As used herein, the term “percentage of sequence identity” may refer to the value determined by comparing two optimally aligned sequences (e.g., nucleic acid sequences, and amino acid sequences) over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity.

Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10.

The National Center for Biotechnology Information (NCBI) Basic Local Alignment Search Tool (BLAST; Altschul et al. (1990)) is available from several sources, including the National Center for Biotechnology Information (Bethesda, Md.), and on the internet, for use in connection with several sequence analysis programs. A description of how to determine sequence identity using this program is available on the internet under the “help” section for BLAST. For comparisons of nucleic acid sequences, the “Blast 2 sequences” function of the BLAST (Blastn) program may be employed using the default parameters. Nucleic acid sequences with even greater similarity to the reference sequences will show increasing percentage identity when assessed by this method.

Operably linked: A first nucleic acid sequence is operably linked with a second nucleic acid sequence when the first nucleic acid sequence is in a functional relationship with the second nucleic acid sequence. For instance, a promoter is operably linked with a coding sequence when the promoter affects the transcription or expression of the coding sequence. When recombinantly produced, operably linked nucleic acid sequences are generally contiguous and, where necessary to join two protein-coding regions, in the same reading frame. However, elements need not be contiguous to be operably linked

Promoter: A region of DNA that generally is located upstream (towards the 5′ region of a gene) that is needed for transcription. Promoters may permit the proper activation or repression of the gene which they control. A promoter may contain specific sequences that are recognized by transcription factors. These factors may bind to the promoter DNA sequences and result in the recruitment of RNA polymerase, an enzyme that synthesizes RNA from the coding region of the gene.

Transformed: A cell is “transformed” by a nucleic acid molecule transduced into the cell when the nucleic acid molecule becomes stably replicated by the cell, either by incorporation of the nucleic acid molecule into the cellular genome or by episomal replication. As used herein, the term “transformation” encompasses all techniques by which a nucleic acid molecule can be introduced into such a cell. Examples include, but are not limited to: transfection with viral vectors; transformation with plasmid vectors; electroporation (Fromm et al. (1986) Nature 319:791-3); lipofection (Felgner et al. (1987) Proc. Natl. Acad. Sci. USA 84:7413-7); microinjection (Mueller et al. (1978) Cell 15:579-85); Agrobacterium-mediated transfer (Fraley et al. (1983) Proc. Natl. Acad. Sci. USA 80:4803-7); direct DNA uptake; whiskers-mediated transformation; and microprojectile bombardment (Klein et al. (1987) Nature 327:70).

Transgene: An exogenous nucleic acid sequence. In one example, a transgene is a gene sequence (e.g., an herbicide-resistance gene), a gene encoding an industrially or pharmaceutically useful compound, or a gene encoding a desirable agricultural trait. In yet another example, the transgene is an antisense nucleic acid sequence, wherein expression of the antisense nucleic acid sequence inhibits expression of a target nucleic acid sequence. A transgene may contain regulatory sequences operably linked to the transgene (e.g., a promoter). In some embodiments, a nucleic acid sequence of interest is a transgene. However, in other embodiments, a polynucleotide sequence of interest is an endogenous nucleic acid sequence, wherein additional genomic copies of the endogenous nucleic acid sequence are desired, or a polynucleotide sequence that is in the antisense orientation with respect to the sequence of a target nucleic acid molecule in the host organism.

Transgenic Event: A transgenic “event” is produced by transformation of plant cells with heterologous DNA, i.e., a nucleic acid construct that includes a transgene of interest, regeneration of a population of plants resulting from the insertion of the transgene into the genome of the plant, and selection of a particular plant characterized by insertion into a particular genome location. The term “event” refers to the original transformant and progeny of the transformant that include the heterologous DNA. The term “event” also refers to progeny produced by a sexual outcross between the transformant and another variety that includes the genomic/transgene DNA. Even after repeated back-crossing to a recurrent parent, the inserted transgene DNA and flanking genomic DNA (genomic/transgene DNA) from the transformed parent is present in the progeny of the cross at the same chromosomal location. The term “event” also refers to DNA from the original transformant and progeny thereof comprising the inserted DNA and flanking genomic sequence immediately adjacent to the inserted DNA that would be expected to be transferred to a progeny that receives inserted DNA including the transgene of interest as the result of a sexual cross of one parental line that includes the inserted DNA (e.g., the original transformant and progeny resulting from selfing) and a parental line that does not contain the inserted DNA.

Vector: A nucleic acid molecule as introduced into a cell, thereby producing a transformed cell. A vector may include nucleic acid sequences that permit it to replicate in the host cell, such as an origin of replication. Examples include, but are not limited to, a plasmid, cosmid, bacteriophage, or virus that carries exogenous DNA into a cell. A vector can also include one or more genes, antisense molecules, and/or selectable marker genes and other genetic elements known in the art. A vector may transduce, transform, or infect a cell, thereby causing the cell to express the nucleic acid molecules and/or proteins encoded by the vector. A vector may optionally include materials to aid in achieving entry of the nucleic acid molecule into the cell (e.g., a liposome, protein coding, etc.).

Unless otherwise specifically explained, all technical and scientific terms used herein have the same meaning as commonly understood by those of ordinary skill in the art to which this disclosure belongs. Definitions of common terms in molecular biology can be found in, for example: Lewin, Genes V, Oxford University Press, 1994 (ISBN 0-19-854287-9); Kendrew et al. (eds.), The Encyclopedia of Molecular Biology, Blackwell Science Ltd., 1994 (ISBN 0-632-02182-9); and Meyers (ed.), Molecular Biology and Biotechnology: A Comprehensive Desk Reference, VCH Publishers, Inc., 1995 (ISBN 1-56081-569-8).

As used herein, the articles, “a,” “an,” and “the” include plural references unless the context clearly and unambiguously dictates otherwise.

### IV. Synthetic Bi-Directional Promoter, RUbi3, and Nucleic Acids Comprising the Same

This disclosure provides nucleic acid molecules comprising a synthetic nucleotide sequence that may function as a bi-directional promoter. In some embodiments, a synthetic bi-directional promoter may be operably linked to one or two polynucleotide sequence(s) of interest. For example, the synthetic Rice Ubiquitin 3 bi-directional promoter may be operably linked to one or two polynucleotide sequence(s) of interest that encode a gene. (e.g., two genes, one on each end of the promoter), so as to regulate transcription of at least one (e.g., one or both) of the nucleotide sequence(s) of interest. In some embodiments, by incorporating a URS from a Rice Ubiquitin 3 promoter in the synthetic Rice Ubiquitin 3 bi-directional promoter, particular expression and regulatory patterns (e.g., such as are exhibited by genes under the control of the Rice Ubiquitin 3 promoter) may be achieved with regard to a polynucleotide sequence of interest that is operably linked to the synthetic Rice Ubiquitin 3 bi-directional promoter.

Some embodiments of the invention are exemplified herein by incorporating a minimal core promoter element from a unidirectional maize ubiquitin-1 gene (ZmUbi1) promoter into a molecular context different from that of the native promoter to engineer a synthetic bidirectional promoter. This minimal core promoter element is referred to herein as “minUbi1P,” and is approximately 200 nt in length. Sequencing and analysis of minUbi1P elements from multiple Zea species and Z. mays genotypes has revealed that functional minUbi1P elements are highly conserved, such that a minUbi1P element may element may preserve its function as an initiator of transcription if it shares, for example, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, and/or at least about 100% sequence identity to the minUbi1P element of SEQ ID NO:2. Characteristics of minUbi1P elements that may be useful in some embodiments of the invention may include, for example, and without limitation, the aforementioned high conservation of nucleotide sequence, the presence of at least one TATA box, and/or the presence of at least one (e.g., two) heat shock consensus element(s). In particular minUbi1P elements, more than one heat shock consensus elements may be overlapping within the minUbi1P sequence.

In embodiments, the process of incorporating a minUbi1P element into a molecular context different from that of a native promoter (i.e., Rice Ubiquitin 3) to engineer a synthetic bi-directional promoter may comprise incorporating the minUbi1P element into a Rice Ubiquitin 3 promoter nucleic acid, while reversing the orientation of the minUbi1P element with respect to the remaining sequence of the Rice Ubiquitin 3 promoter. Thus, a synthetic Rice Ubiquitin 3 bi-directional promoter may comprise a minUbi1P minimal core promoter element located 3′ of, and in reverse orientation with respect to, a Rice Ubiquitin 3 promoter nucleotide sequence, such that it may be operably linked to a nucleotide sequence of interest located 3′ of the Rice Ubiquitin 3 promoter nucleotide sequence. For example, the minUbi1P element may be incorporated at the 3′ end of a Rice Ubiquitin 3 promoter in reverse orientation.

A synthetic bi-directional Rice Ubiquitin 3 promoter may also comprise one or more additional sequence elements in addition to a minUbi1P element and elements of a native Rice Ubiquitin 3 promoter. In some embodiments, a synthetic bi-directional Rice Ubiquitin 3 promoter may comprise a promoter URS, an exon (e.g., a leader or signal peptide), an intron, a spacer sequence, and/or combinations of one or more of any of the foregoing. For example and without limitation, a synthetic bi-directional Rice Ubiquitin 3 promoter may comprise a URS sequence from a Rice Ubiquitin 3 or ZmUbi1 promoter, an intron from a Rice Ubiquitin 3 or ZmUbi1 gene, an exon encoding a leader peptide from an Rice Ubiquitin 3 or ZmUbi1 gene, an intron from an Rice Ubiquitin 3 or ZmUbi1 gene, and combinations of these.

A synthetic bi-directional Rice Ubiquitin 3 promoter may also comprise one or more additional sequence elements in addition to a minUBi1P element and elements of a native promoter Rice Ubiquitin 3 including the minUbi1P. In some embodiments, a synthetic bi-directional Rice Ubiquitin 3 promoter may comprise a promoter URS, an exon (e.g., a leader or signal peptide), an intron, a spacer sequence, and or combinations of one or more of any of the foregoing. For example and without limitation, a synthetic bi-directional Rice Ubiquitin 3 promoter may comprise a URS sequence from a Zea mays Ubiquitin 1 promoter, an intron from a ADH gene, an exon encoding a leader peptide from an Zea mays Ubiquitin gene, an intron from an Zea mays Ubiquitin gene, and combinations of these.

In some embodiments of a promoter comprising a promoter URS, the URS may be selected to confer particular regulatory properties on the synthetic promoter. Known promoters vary widely in the type of control they exert on operably linked genes (e.g., environmental responses, developmental cues, and spatial information), and a URS incorporated into a heterologous promoter typically maintains the type of control the URS exhibits with regard to its native promoter and operably linked gene(s). Langridge et al. (1989), supra. Examples of eukaryotic promoters that have been characterized and may contain a URS comprised within a synthetic bi-directional Rice Ubiquitin 3 promoter according to some embodiments include, for example and without limitation: those promoters described in U.S. Pat. No. 6,437,217 (maize RS81 promoter); U.S. Pat. No. 5,641,876 (rice actin promoter); U.S. Pat. No. 6,426,446 (maize RS324 promoter); U.S. Pat. No. 6,429,362 (maize PR-1 promoter); U.S. Pat. No. 6,232,526 (maize A3 promoter); U.S. Pat. No. 6,177,611 (constitutive maize promoters); U.S. Pat. No. 6,433,252 (maize L3 oleosin promoter); U.S. Pat. No. 6,429,357 (rice actin 2 promoter, and rice actin 2 intron); U.S. Pat. No. 5,837,848 (root-specific promoter); U.S. Pat. No. 6,294,714 (light-inducible promoters); U.S. Pat. No. 6,140,078 (salt-inducible promoters); U.S. Pat. No. 6,252,138 (pathogen-inducible promoters); U.S. Pat. No. 6,175,060 (phosphorous deficiency-inducible promoters); U.S. Pat. No. 6,388,170 (bi-directional promoters); U.S. Pat. No. 6,635,806 (gamma-coixin promoter); and U.S. patent application Ser. No. 09/757,089 (maize chloroplast aldolase promoter).

Additional exemplary prokaryotic promoters include the nopaline synthase (NOS) promoter (Ebert et al. (1987) Proc. Natl. Acad. Sci. USA 84(16):5745-9); the octopine synthase (OCS) promoter (which is carried on tumor-inducing plasmids of Agrobacterium tumefaciens); the caulimovirus promoters such as the cauliflower mosaic virus (CaMV) 19S promoter (Lawton et al. (1987) Plant Mol. Biol. 9:315-24); the CaMV 35S promoter (Odell et al. (1985) Nature 313:810-2); the figwort mosaic virus 35S-promoter (Walker et al. (1987) Proc. Natl. Acad. Sci. USA 84(19):6624-8); the sucrose synthase promoter (Yang and Russell (1990) Proc. Natl. Acad. Sci. USA 87:4144-8); the R gene complex promoter (Chandler et al. (1989) Plant Cell 1:1175-83); CaMV35S (U.S. Pat. Nos. 5,322,938, 5,352,605, 5,359,142, and 5,530,196); FMV35S (U.S. Pat. Nos. 6,051,753, and 5,378,619); a PC1SV promoter (U.S. Pat. No. 5,850,019); the SCP1 promoter (U.S. Pat. No. 6,677,503); and Agrobacterium tumefaciens Nos promoters (GenBank Accession No. V00087; Depicker et al. (1982) J. Mol. Appl. Genet. 1:561-73; Bevan et al. (1983) Nature 304:184-7), and the like.

In some embodiments, a synthetic Rice Ubiquitin 3 bi-directional promoter may further comprise an exon. For example, it may be desirable to target or traffic a polypeptide encoded by a polynucleotide sequence of interest operably linked to the promoter to a particular subcellular location and/or compartment. In these and other embodiments, a coding sequence (exon) may be incorporated into a nucleic acid molecule between the remaining synthetic Rice Ubiquitin 3 bi-directional promoter sequence and a nucleotide sequence encoding a polypeptide. These elements may be arranged according to the discretion of the skilled practitioner such that the synthetic Rice Ubiquitin 3 bi-directional promoter promotes the expression of a polypeptide (or one or both of two polypeptide-encoding sequences that are operably linked to the promoter) comprising the peptide encoded by the incorporated coding sequence in a functional relationship with the remainder of the polypeptide. In particular examples, an exon encoding a leader, transit, or signal peptide (e.g., a Zea mays Ubi1 leader peptide) may be incorporated.

Peptides that may be encoded by an exon incorporated into a synthetic Rice Ubiquitin 3 bi-directional promoter include, for example and without limitation: a Ubiquitin (e.g., Zea mays Ubi1) leader peptide, a chloroplast transit peptide (CTP) (e.g., the A. thaliana EPSPS CTP (Klee et al. (1987) Mol. Gen. Genet. 210:437-42), and the Petunia hybrida EPSPS CTP (della-Cioppa et al. (1986) Proc. Natl. Acad. Sci. USA 83:6873-7)), as exemplified for the chloroplast targeting of dicamba monooxygenase (DMO) in International PCT Publication No. WO 2008/105890.

Introns may also be incorporated in a synthetic Rice Ubiquitin 3 bi-directional promoter in some embodiments of the invention, for example, between the remaining synthetic Rice Ubiquitin 3 bi-directional promoter sequence and a polynucleotide sequence of interest that is operably linked to the promoter. In some examples, an intron incorporated into a synthetic Rice Ubiquitin 3 bi-directional promoter may be, without limitation, a 5′ UTR that functions as a translation leader sequence that is present in a fully processed mRNA upstream of the translation start sequence (such a translation leader sequence may affect processing of a primary transcript to mRNA, mRNA stability, and/or translation efficiency). Examples of translation leader sequences include maize and petunia heat shock protein leaders (U.S. Pat. No. 5,362,865), plant virus coat protein leaders, plant rubisco leaders, and others. See, e.g., Turner and Foster (1995) Molecular Biotech. 3(3):225-36. Non-limiting examples of 5′ UTRs include GmHsp (U.S. Pat. No. 5,659,122), PhDnaK (U.S. Pat. No. 5,362,865), AtAnt1, TEV (Carrington and Freed (1990) J. Virol. 64:1590-7), and AGRtunos (GenBank Accession No. V00087, and Bevan et al. (1983) Nature 304:184-7). In particular examples, a Zea mays Ubiquitin 1 intron may be incorporated in a synthetic Rice Ubiquitin-3 bi-directional promoter.

Additional sequences that may optionally be incorporated into a synthetic Rice Ubiquitin-3 bi-directional promoter include, for example and without limitation: 3′ non-translated sequences, 3′ transcription termination regions, and polyadenylation regions. These are genetic elements located downstream of a polynucleotide sequence of interest (e.g., a sequence of interest that is operably linked to a synthetic Rice Ubiquitin-3 bi-directional promoter), and include polynucleotides that provide polyadenylation signal, and/or other regulatory signals capable of affecting transcription, mRNA processing, or gene expression. A polyadenylation signal may function in plants to cause the addition of polyadenylate nucleotides to the 3′ end of a mRNA precursor. The polyadenylation sequence may be derived from the natural gene, from a variety of plant genes, or from T-DNA genes. A non-limiting example of a 3′ transcription termination region is the nopaline synthase 3′ region (nos 3′; Fraley et al. (1983) Proc. Natl. Acad. Sci. USA 80:4803-7). An example of the use of different 3′ nontranslated regions is provided in Ingelbrecht et al., (1989) Plant Cell 1:671-80. Non-limiting examples of polyadenylation signals include one from a Pisum sativum RbcS2 gene (Ps.RbcS2-E9; Coruzzi et al. (1984) EMBO J. 3:1671-9) and Agrobacterium tumefaciens Nos gene (GenBank Accession No. E01312).

In some embodiments, a synthetic Rice Ubiquitin-3 bi-directional promoter comprises one or more nucleotide sequence(s) that facilitate targeting of a nucleic acid comprising the promoter to a particular locus in the genome of a target organism. For example, one or more sequences may be included that are homologous to segments of genomic DNA sequence in the host (e.g., rare or unique genomic DNA sequences). In some examples, these homologous sequences may guide recombination and integration of a nucleic acid comprising a synthetic Rice Ubiquitin-3 bi-directional promoter at the site of the homologous DNA in the host genome. In particular examples, a synthetic Rice Ubiquitin-3 bi-directional promoter comprises one or more nucleotide sequences that facilitate targeting of a nucleic acid comprising the promoter to a rare or unique location in a host genome utilizing engineered nuclease enzymes that recognize sequence at the rare or unique location and facilitate integration at that rare or unique location. Such a targeted integration system employing zinc-finger endonucleases as the nuclease enzyme is described in U.S. patent application Ser. No. 13/011,735, the contents of the entirety of which are incorporated herein by this reference.

In other embodiments, the disclosure further includes as an embodiment the polynucleotide sequence of interest comprising a trait. The trait can be an insecticidal resistance trait, herbicide tolerance trait, nitrogen use efficiency trait, water use efficiency trait, nutritional quality trait, DNA binding trait, selectable marker trait, and any combination thereof.

In further embodiments the traits are integrated within the transgenic plant cell as a transgenic event. In additional embodiments, the transgenic event produces a commodity product. Accordingly, a composition is derived from transgenic plant cells of the subject disclosure, wherein said composition is a commodity product selected from the group consisting of meal, flour, protein concentrate, or oil. In further embodiments, commodity products produced by transgenic plants derived from transformed plant cells are included, wherein the commodity products comprise a detectable amount of a nucleic acid sequence of the invention. In some embodiments, such commodity products may be produced, for example, by obtaining transgenic plants and preparing food or feed from them. Commodity products comprising one or more of the nucleic acid sequences of the invention includes, for example and without limitation: meals, oils, crushed or whole grains or seeds of a plant, and any food product comprising any meal, oil, or crushed or whole grain of a recombinant plant or seed comprising one or more of the nucleic acid sequences of the invention. The detection of one or more of the sequences of the invention in one or more commodity or commodity products is de facto evidence that the commodity or commodity product is produced from a transgenic plant designed to express one or more agronomic traits.

Nucleic acids comprising a synthetic Rice Ubiquitin-3 bi-directional promoter may be produced using any technique known in the art, including, for example and without limitation: RCA, PCR amplification, RT-PCR amplification, OLA, and SNuPE. These and other equivalent techniques are well known to those of skill in the art, and are further described in detail in, for example and without limitation: Sambrook et al. Molecular Cloning: A Laboratory Manual, 3^{rd} Ed., Cold Spring Harbor Laboratory, 2001; and Ausubel et al. Current Protocols in Molecular Biology, John Wiley & Sons, 1998. All of the references cited above, including both of the foregoing manuals, are incorporated herein by this reference in their entirety, including any drawings, figures, and/or tables provided therein.

### V. Delivery to a Cell of a Nucleic Acid Molecule Comprising Synthetic Bi-Directional Promoter, RUbi3

The present disclosure also provides methods for transforming a cell with a nucleic acid molecule comprising a synthetic Rice Ubiquitin-3 bi-directional promoter. Any of the large number of techniques known in the art for introduction of nucleic acid molecules into plants may be used to transform a plant with a nucleic acid molecule comprising a synthetic Rice Ubiquitin-3 bi-directional promoter according to some embodiments, for example, to introduce one or more synthetic Rice Ubiquitin-3 bi-directional promoters into the host plant genome, and/or to further introduce one or more polynucleotides of interest operably linked to the promoter.

Suitable methods for transformation of plants include any method by which DNA can be introduced into a cell, for example and without limitation: electroporation (see, e.g., U.S. Pat. No. 5,384,253), microprojectile bombardment (see, e.g., U.S. Pat. Nos. 5,015,580, 5,550,318, 5,538,880, 6,160,208, 6,399,861, and 6,403,865), Agrobacterium-mediated transformation (see, e.g., U.S. Pat. Nos. 5,635,055, 5,824,877, 5,591,616, 5,981,840, and 6,384,301), and protoplast transformation (see, e.g., U.S. Pat. No. 5,508,184). Through the application of techniques such as the foregoing, the cells of virtually any plant species may be stably transformed, and these cells may be developed into transgenic plants by techniques known to those of skill in the art. For example, techniques that may be particularly useful in the context of cotton transformation are described in U.S. Pat. Nos. 5,846,797, 5,159,135, 5,004,863, and 6,624,344; techniques for transforming Brassica plants in particular are described, for example, in U.S. Pat. No. 5,750,871; techniques for transforming soya are described, for example, in U.S. Pat. No. 6,384,301; and techniques for transforming maize are described, for example, in U.S. Pat. Nos. 7,060,876 and 5,591,616, and International PCT Publication WO 95/06722.

After effecting delivery of an exogenous nucleic acid to a recipient cell, the transformed cell is generally identified for further culturing and plant regeneration. In order to improve the ability to identify transformants, one may desire to employ a selectable or screenable marker gene with the transformation vector used to generate the transformant. In this case, the potentially transformed cell population can be assayed by exposing the cells to a selective agent or agents, or the cells can be screened for the desired marker gene trait.

Cells that survive the exposure to the selective agent, or cells that have been scored positive in a screening assay, may be cultured in media that supports regeneration of plants. In some embodiments, any suitable plant tissue culture media (e.g., MS and N6 media) may be modified by including further substances, such as growth regulators. Tissue may be maintained on a basic media with growth regulators until sufficient tissue is available to begin plant regeneration efforts, or following repeated rounds of manual selection, until the morphology of the tissue is suitable for regeneration (e.g., at least 2 weeks), then transferred to media conducive to shoot formation. Cultures are transferred periodically until sufficient shoot formation has occurred. Once shoots are formed, they are transferred to media conducive to root formation. Once sufficient roots are formed, plants can be transferred to soil for further growth and maturity.

To confirm the presence of the desired nucleic acid molecule comprising a synthetic Rice Ubiquitin-3 bi-directional promoter in the regenerating plants, a variety of assays may be performed. Such assays include, for example: molecular biological assays, such as Southern and Northern blotting and PCR; biochemical assays, such as detecting the presence of a protein product, e.g., by immunological means (ELISA and/or Western blots) or by enzymatic function; plant part assays, such as leaf or root assays; and analysis of the phenotype of the whole regenerated plant.

Targeted integration events may be screened, for example, by PCR amplification using, e.g., oligonucleotide primers specific for nucleic acid molecules of interest. PCR genotyping is understood to include, but not be limited to, polymerase-chain reaction (PCR) amplification of genomic DNA derived from isolated host plant callus tissue predicted to contain a nucleic acid molecule of interest integrated into the genome, followed by standard cloning and sequence analysis of PCR amplification products. Methods of PCR genotyping have been well described (see, e.g., Rios et al. (2002) Plant J. 32:243-53), and may be applied to genomic DNA derived from any plant species or tissue type, including cell cultures. Combinations of oligonucleotide primers that bind to both target sequence and introduced sequence may be used sequentially or multiplexed in PCR amplification reactions. Oligonucleotide primers designed to anneal to the target site, introduced nucleic acid sequences, and/or combinations of the two may be produced. Thus, PCR genotyping strategies may include, for example and without limitation: amplification of specific sequences in the plant genome, amplification of multiple specific sequences in the plant genome, amplification of non-specific sequences in the plant genome, and combinations of any of the foregoing. One skilled in the art may devise additional combinations of primers and amplification reactions to interrogate the genome. For example, a set of forward and reverse oligonucleotide primers may be designed to anneal to nucleic acid sequence(s) specific for the target outside the boundaries of the introduced nucleic acid sequence.

Forward and reverse oligonucleotide primers may be designed to anneal specifically to an introduced nucleic acid molecule, for example, at a sequence corresponding to a coding region within a polynucleotide sequence of interest comprised therein, or other parts of the nucleic acid molecule. These primers may be used in conjunction with the primers described above. Oligonucleotide primers may be synthesized according to a desired sequence, and are commercially available (e.g., from Integrated DNA Technologies, Inc., Coralville, Iowa). Amplification may be followed by cloning and sequencing, or by direct sequence analysis of amplification products. One skilled in the art might envision alternative methods for analysis of amplification products generated during PCR genotyping. In one embodiment, oligonucleotide primers specific for the gene target are employed in PCR amplifications.

### VI. Cells, Cell Cultures, Tissues, and Organisms Comprising Synthetic Bi-Directional Promoter, RUbi3

Some embodiments of the present invention also provide cells comprising a synthetic Rice Ubiquitin-3 bi-directional promoter, for example, as may be present in a nucleic acid construct. In particular examples, a synthetic Rice Ubiquitin-3 bi-directional promoter according to some embodiments may be utilized as a regulatory sequence to regulate the expression of transgenes in plant cells and plants. In some such examples, the use of a synthetic bi-directional RUbi3 promoter operably linked to a polynucleotide sequence of interest (e.g., a transgene) may reduce the number of homologous promoters needed to regulate expression of a given number of polynucleotide sequences of interest, and/or reduce the size of the nucleic acid construct(s) required to introduce a given number of nucleotide sequences of interest. Furthermore, use of a synthetic Rice Ubiquitin-3 bi-directional promoter may allow co-expression of two operably linked nucleotide sequence of interest under the same conditions (i.e., the conditions under which the RUbi3 promoter is active). Such examples may be particularly useful, e.g., when the two operably linked polynucleotide sequences of interest each contribute to a single trait in a transgenic host comprising the nucleotide sequences of interest, and co-expression of the nucleotide sequences of interest advantageously impacts expression of the trait in the transgenic host.

In some embodiments, a transgenic plant comprising one or more synthetic Rice Ubiquitin-3 bi-directional promoter(s) and/or nucleotide sequence(s) of interest may have one or more desirable traits conferred (e.g., introduced, enhanced, or contributed to) by expression of the nucleotide sequence(s) of interest in the plant. Such traits may include, for example and without limitation: resistance to insects, other pests, and disease-causing agents; tolerance to herbicides; enhanced stability, yield, or shelf-life; environmental tolerances; pharmaceutical production; industrial product production; and nutritional enhancements. In some examples, a desirable trait may be conferred by transformation of a plant with a nucleic acid molecule comprising a synthetic Rice Ubiquitin-3 bi-directional promoter operably linked to a polynucleotide sequence of interest. In some examples, a desirable trait may be conferred to a plant produced as a progeny plant via breeding, which trait may be conferred by one or more nucleotide sequences of interest operably linked to a synthetic Rice Ubiquitin-3 bi-directional promoter that is/are passed to the plant from a parent plant comprising a nucleotide sequence of interest operably linked to a synthetic Rice Ubiquitin-3 bi-directional promoter.

A transgenic plant according to some embodiments may be any plant capable of being transformed with a nucleic acid molecule of the invention, or of being bred with a plant transformed with a nucleic acid molecule of the invention. Accordingly, the plant may be a dicot or monocot. Non-limiting examples of dicotyledonous plants for use in some examples include: alfalfa, beans, broccoli, cabbage, canola, carrot, cauliflower, celery, Chinese cabbage, cotton, cucumber, eggplant, lettuce, melon, pea, pepper, peanut, potato, pumpkin, radish, rapeseed, spinach, soybean, squash, sugarbeet, sunflower, tobacco, tomato, and watermelon. Non-limiting examples of monocotyledonous plants for use in some examples include: Brachypodium, corn, onion, rice, sorghum, wheat, rye, millet, sugarcane, oat, triticale, switchgrass, and turfgrass.

In some embodiments, a transgenic plant may be used or cultivated in any manner, wherein presence a synthetic Rice Ubiquitin-3 bi-directional promoter and/or operably linked polynucleotide sequence of interest is desirable. Accordingly, such transgenic plants may be engineered to, inter alia, have one or more desired traits or transgenic events, by being transformed with nucleic acid molecules according to the invention, and may be cropped or cultivated by any method known to those of skill in the art.

The following examples are provided to illustrate certain particular features and/or embodiments. The examples should not be construed to limit the disclosure to the particular features or embodiments exemplified.

### EXAMPLES

### Example 1

### Design of Bi-Directional Promoter

A bi-directional promoter that contained gene regulatory elements from the Zea mays Ubiquitin 1 (ZmUbi1) and the Rice Ubiquitin 3 (Rubi3) promoters was designed and is presented as SEQ ID NO:1. This bi-directional promoter contains sequence of the partial ZmUbi1 promoter (base pairs 1-1,313; SEQ ID NO:8) fused in reverse complimentary orientation to the 5′ end of the full length Rubi3 promoter (base pairs 1,314-3,303; SEQ ID NO:9). The components of the partial ZmUbi1 promoter contain a 215 bp region of the ZmUbi1 minimal core promoter (underlined font at base pairs 1,099-1,313; SEQ ID NO:2), the ZmUbi1 5′ untranslated region (bold font at base pairs 1,016-1,097; SEQ ID NO:3), and the ZmUbi1 intron (lower case font at base pairs 1-1,015, SEQ ID NO:4). The components of the full length Rice Ubi3 promoter contain an upstream and core promoter region (italics font at base pairs 1,314-2,096; SEQ ID NO:7), the Rice Ubi3 5′ untranslated region (bold and underlined font at base pars 2,097-2,163; SEQ ID NO:5) and the Rice Ubi3 intron (underlined and lower case font at base pairs 2,164-3,303; SEQ ID NO:6).

SEQ ID NO: 1:
tgcagaagtaacaccaaacaacagggtgagcatcgacaaaagaaa
cagtaccaagcaaataaatagcgtatgaaggcagggctaaaaaaa
tccacatatagctgctgcatatgccatcatccaagtatatcaaga
tcgaaataattataaaacatacttgtttattataatagataggta
ctcaaggttagagcatatgaatagatgctgcatatgccatcatgt
atatgcatcagtaaaacccacatcaacatgtatacctatcctaga
tcgatatttccatccatcttaaactcgtaactatgaagatgtatg
acacacacatacagttccaaaattaataaatacaccaggtagttt
gaaacagtattctactccgatctagaacgaatgaacgaccgccca
accacaccacatcatcacaaccaagcgaacaaaaagcatctctgt
atatgcatcagtaaaacccgcatcaacatgtatacctatcctaga
tcgatatttccatccatcatcttcaattcgtaactatgaatatgt
atggcacacacatacagatccaaaattaataaatccaccaggtag
tttgaaacagaattctactccgatctagaacgaccgcccaaccag
accacatcatcacaaccaagacaaaaaaaagcatgaaaagatgac
ccgacaaacaagtgcacggcatatattgaaataaaggaaaagggc
aaaccaaaccctatgcaacgaaacaaaaaaaatcatgaaatcgat
cccgtctgcggaacggctagagccatcccaggattccccaaagag
aaacactggcaagttagcaatcagaacgtgtctgacgtacaggtc
gcatccgtgtacgaacgctagcagcacggatctaacacaaacacg
gatctaacacaaacatgaacagaagtagaactaccgggccctaac
catgcatggaccggaacgccgatctagagaaggtagagagggggg
gggggggggaggacgagcggcgtacCTTGAAGCGGAGGTGCCGAC
GGGTGGATTTGGGGGAGATCTGGTTGTGTGTGTGTGCGCTCCGAA
CAACACGAGGTTGGGGAAAGAGGGTGTGGAGGGGGTGTCTATTTA
TTACGGCGGGCGAGGAAGGGAAAGCGAAGGAGCGGTGGGAAAGGA
ATCCCCCGTAGCTGCCGGTGCCGTGAGAGGAGGAGGAGGCCGCCT
GCCGTGCCGGCTCACGTCTGCCGCTCCGCCACGCAATTTCTGGAT
GCCGACAGCGGAGCAAGTCCAACGGTGGAGCGGAACTCTCGAGAG
GGGTCCAGGATGTGAAGAACAGGTAAATCACGCAGAAGAACCCAT
CTCTGATAGCAGCTATCGATTAGAACAACGAATCCATATTGGGTC
CGTGGGAAATACTTACTGCACAGGAAGGGGGCGATCTGACGAGGC
CCCGCCACCGGCCTCGACCCGAGGCCGAGGCCGACGAAGCGCCGG
CGAGTACGGCGCCGCGGCGGCCTCTGCCCGTGCCCTCTGCGCGTG
GGAGGGAGAGGCCGCGGTGGTGGGGGCGCGCGCGCGCGCGCGCGC
AGCTGGTGCGGCGGCGCGGGGGTCAGCCGCCGAGCCGGCGGCGAC
GGAGGAGCAGGGCGGCGTGGACGCGAACTTCCGATCGGTTGGTCA
GAGTGCGCGAGTTGGGCTTAGCCAATTAGGTCTCAACAATCTATT
GGGCCGTAAAATTCATGGGCCCTGGTTTGTCTAGGCCCAATATCC
CGTTCATTTCAGCCCACAAATATTTCCCCAGAGGATTATTAAGGC
CCACACGCAGCTTATAGCAGATCAAGTACGATGTTTCCTGATCGT
TGGATCGGAAACGTACGGTCTTGATCAGGCATGCCGACTTCGTCA
AAGAGAGGCGGCATGACCTGACGCGGAGTTGGTTCCGGGCACCGT
CTGGATGGTCGTACCGGGACCGGACACGTGTCGCGCCTCCAACTA
CATGGACACGTGTGGTGCTGCCATTGGGCCGTACGCGTGGCGGTG
ACCGCACCGGATGCTGCCTCGCACCGCCTTGCCCACGCTTTATAT
AGAGAGGTTTTCTCTCCATTAATCGCATAGCGAGTCGAATCGACC
GAAGGGGAGGGGGAGCGAAGCTTTGCGTTCTCTAATCGCCTCGTC
AAGgtaactaatcaatcacctcgtcctaatcctcgaatctctcgt
ggtgcccgtctaatctcgcgattttgatgctcgtggtggaaagcg
taggaggatcccgtgcgagttagtctcaatctctcagggtttcgt
gcgattttagggtgatccacctcttaatcgagttacggtttcgtg
cgattttagggtaatcctcttaatctctcattgatttagggtttc
gtgagaatcgaggtagggatctgtgttatttatatcgatctaata
gatggattggttttgagattgttctgtcagatggggattgtttcg
atatattaccctaatgatgtgtcagatggggattgtttcgatata
ttaccctaatgatgtgtcagatggggattgtttcgatatattacc
ctaatgatggataataagagtagttcacagttatgttttgatcct
gccacatagtttgagttttgtgatcagatttagtttcacttattt
gtgcttagttcggatgggattgttctgatattgttccaatagatg
aatagctcgttaggttaaaatctttaggttgagttaggcgacaca
tagtttatttcctctggatttggattggaattgtgttcttagttt
ttttcccctggatttggattggaattgtgtggagctgggttagag
aattacatctgtatcgtgtacacctacttgaactgtagagcttgg
gttctaaggtcaatttaatctgtattgtatctggctctttgccta
gttgaactgtagtgctgatgttgtactgtgtttttttacccgttt
tatttgctttactcgtgcaaatcaaatctgtcagatgctagaact
aggtggctttattctgtgttcttacatagatctgttgtcctgtag
ttacttatgtcagttttgttattatctgaagatatttttggttgt
tgcttgttgatgtggtgtgagctgtgagcagcgctcttatgatta
atgatgctgtccaattgtagtgtaatatgatgtgattgatatgtt
catctattttgagctgacagtaccgatatcgtaggatctggtgcc
aacttattctccagctgcttttttttacctatgttaattccaatc
ctttcttgcctcttccag

### Example 2

### Plant Transformation Constructs

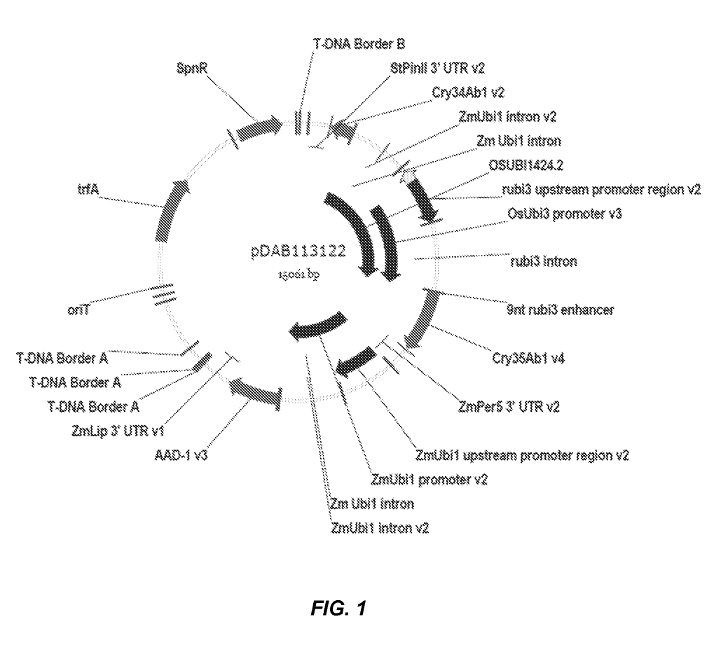

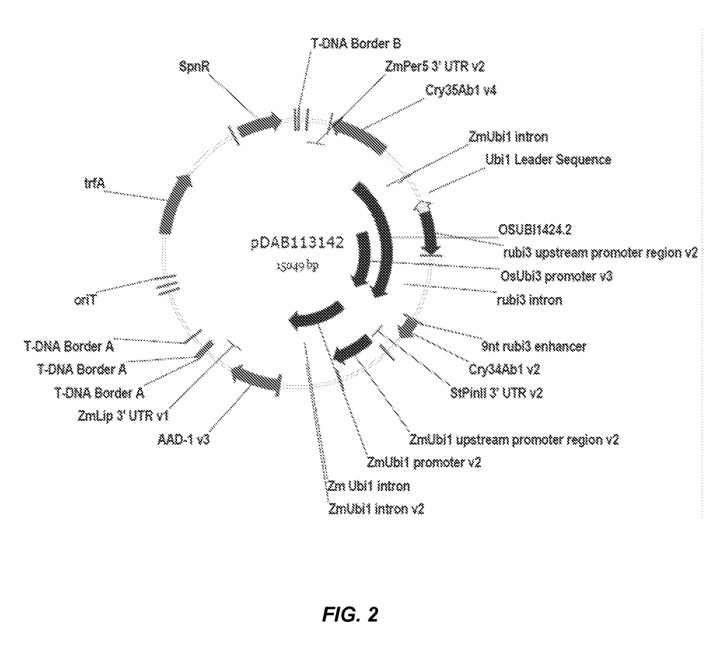

Plant transformation constructs were designed to test the expression of the bi-directional promoter in planta. The final bi-directional promoter construct were generated by inserting a Zea mays Ubiquitin 1 minimal promoter driving one reporter gene upstream and in reverse complimentary orientation of the primary Rice Ubiquitin 3 promoter driving the second reporter gene. Two binary plasmids, pDAB113122 (FIG. 1; SEQ ID NO:22) and pDAB113142 (FIG. 2; SEQ ID NO:23) were built to contain the novel bi-directional promoter of SEQ ID NO:1 driving both the Cry34Ab1 (Li H, Olson M, Lin G, Hey T, Tan S Y, Narva K E (2013) Bacillus thuringiensis Cry34Ab1/Cry35Ab1 interactions with western corn rootworm midgut membrane binding sites. PLoS One 8: e53079) and Cry 35Ab1 (Li H, Olson M, Lin G, Hey T, Tan S Y, Narva K E (2013) Bacillus thuringiensis Cry34Ab1/Cry35Ab1 interactions with western corn rootworm midgut membrane binding sites. PLoS One 8: e53079) transgenes and terminated by either the Solanum tuberosum StPinII 3′ UTR (An et al., (1989) Plant Cell 1; 115-22) or the Zea mays Per5 3′ UTR (U.S. Pat. No. 6,699,984). The resulting constructs contained a single bi-directional promoter of SEQ ID NO:1 that drove two different transgenes, which were operably linked to the 5′ and 3′ end of the bi-directional promoter. The constructs also includes a selectable marker gene expression cassette that contained the Zea mays Ubiquitin 1 promoter (Christensen et al., (1992) Plant Molecular Biology 18; 675-689), the aad-1 gene (U.S. Pat. No. 7,838,733), and the Zea mays Lipase 3′UTR (U.S. Pat. No. 7,179,902).

### Example 3

### Maize Transformation

The above-described constructs were used to transform maize cells. Experimental constructs were transformed into maize via Agrobacterium-mediated transformation of immature embryos isolated from the inbred line, Zea mays c.v. B104. The method used is similar to those published by Ishida et al., (1996) Nature Biotechnol 14:745-750 and Frame et al., (2006) Plant Cell Rep 25: 1024-1034, but with several modifications and improvements to make the method amenable to high-throughput transformation. An example of a method used to produce a number of transgenic events in maize is given herein.

Transformation of Agrobacterium tumefaciens

The binary expression vectors were transformed into Agrobacterium tumefaciens strain DAt13192 (RecA deficient ternary strain) (Int'l. Pat. Pub. No. WO2012016222). Bacterial colonies were selected and binary plasmid DNA was isolated and confirmed via restriction enzyme digestion.

Agrobacterium Culture Initiation

Agrobacterium cultures were streaked from glycerol stocks onto AB minimal medium and incubated at 20° C. in the dark for 3 days. Agrobacterium cultures were then streaked onto a plate of YEP medium and incubated at 20° C. in the dark for 1 day.

On the day of an experiment, a mixture of Inoculation medium and acetosyringone was prepared in a volume appropriate to the number of constructs in the experiment. Inoculation medium was pipetted into a sterile, disposable, 250 ml flask. A 1 M stock solution of acetosyringone in 100% dimethyl sulfoxide was added to the flask containing Inoculation medium in a volume appropriate to make a final acetosyringone concentration of 200 μM.

For each construct, 1-2 loops of Agrobacterium from the YEP plate were suspended in 15 ml of the inoculation medium/acetosyringone mixture inside a sterile, disposable, 50 ml centrifuge tube and the optical density of the solution at 600 nm (O.D.₆₀₀) was measured in a spectrophotometer. The suspension was then diluted down to 0.25-0.35 O.D.₆₀₀ using additional Inoculation medium/acetosyringone mixture. The tube of Agrobacterium suspension was then placed horizontally on a platform shaker set at about 75 rpm at room temperature for between 1 and 4 hours before use.

Ear Sterilization and Embryo Isolation

Ears from Zea mays cultivar B104 were harvested 10-12 days post pollination. Harvested ears were de-husked and surface-sterilized by immersion in a 20% solution of commercial bleach (Ultra CLOROX® Germicidal Bleach, 6.15% sodium hypochlorite) and two drops of Tween 20, for 20 minutes, followed by three rinses in sterile, deionized water inside a laminar flow hood. Immature zygotic embryos (1.8-2.2 mm long) were aseptically excised from each ear and distributed into one or more micro-centrifuge tubes containing 2.0 ml of Agrobacterium suspension into which 2 μl of 10% BREAK-THRU® S233 surfactant (Evonik Industries AG, Essen, Germany) had been added. Transformation proceeded according to the method described in U.S. Patent Application Publication No. US 2013/0157369 A1.

### Example 4

### Molecular Confirmation

Putative transgenic maize plants were sampled at the V2-3 leaf stage for transgene presence using cry34Ab1 and aad-1 quantitative PCR assays. Total DNA was extracted from the leaf samples, using MAGATTRACT® DNA extraction kit (Qiagen) as per manufacturer's instructions.

To detect the genes of interest, gene-specific DNA fragments were amplified with TAQMAN® primer/probe sets containing a FAM-labeled fluorescent probe for the cry34Ab1 gene and a HEX-labeled fluorescent probe for the endogenous invertase reference gene control. The following primers were used for the cry34Ab1 and invertase endogenous reference gene amplifications. The primer sequences were as follows:

Cry34Ab1 Primers/Probes:

Forward Primer: TQ.8v6.1.F:
(SEQ ID NO: 10)
GCCATACCCTCCAGTTG
Reverse Primer: TQ.8v6.1.R:
(SEQ ID NO: 11)
GCCGTTGATGGAGTAGTAGATGG
Probe: TQ.8v6.1.MGB.P:
(SEQ ID NO: 12)
5′-/56-FAM/CCGAATCCAACGGCTTCA/MGB

Invertase Primers:

Forward Primer: InvertaseF:
(SEQ ID NO: 13)
TGGCGGACGACGACTTGT
Reverse Primer: InvertaseR:
(SEQ ID NO: 14)
AAAGTTTGGAGGCTGCCGT
InvertaseProbe:
(SEQ ID NO: 15)
5′-/5HEX/CGAGCAGACCGCCGTGTACTT/3BHQ_1/-3′

Next, the PCR reactions were carried out in a final volume of 10 μl reaction containing 5 μl of Roche LIGHTCYCLER® 480 Probes Master Mix (Roche Applied Sciences, Indianapolis, Ind.); 0.4 μl each of TQ.8v6.1.F, TQ.8v6.1.R, Invertase F, and InvertaseR primers from 10 μM stocks to a final concentration of 400 nM; 0.4 μl each of TQ.8v6.1.MGB.P and Invertase Probes from 5 μM stocks to a final concentration of 200 nM, 0.1 μl of 10% polyvinylpyrrolidone (PVP) to final concentration of 0.1%; 2 μl of 10 ng/μl genomic DNA and 0.5 μl water. The DNA was amplified in a Roche LIGHTCYCLER® 480 System under the following conditions: 1 cycle of 95° C. for 10 minutes; 40 cycles of the following 3-steps: 95° C. for 10 seconds; 58° C. for 35 seconds and 72° C. for 1 second, and a final cycle of 4° C. for 10 seconds. Cry34Ab1 copy number was determined by comparison of Target (gene of interest)/Reference (Invertase gene) values for unknown samples (output by the LIGHTCYCLER® 480) to Target/Reference values of cry34Ab1 copy number controls. In addition, contamination by inadvertent integration of the binary vector plasmid backbone was detected by a Hydrolysis Probe assay specific for the Spectinomycin (Spec) resistance gene borne on the binary vector backbone.

The detection of the aad-1 gene was carried out as described above for the cry34Ab1 gene using the invertase endogenous reference gene. The aad-1 primer sequences were as follows:

AAD1 Forward Primer:
(SEQ ID NO: 16)
TGTTCGGTTCCCTCTACCAA
AAD1 Reverse Primer:
(SEQ ID NO: 17)
CAACATCCATCACCTTGACTGA
AAD1 Probe:
(SEQ ID NO: 18)
5′-FAM/CACAGAACCGTCGCTTCAGCAACA-MGB/BHQ-3′

The detection of the spec gene that is present in the binary backbone used to produce the transgenic plants was assayed with primers of the following sequences:

SPC1a:
(SEQ ID NO: 19)
CTTAGCTGGATAACGCCAC
SPC1s:
(SEQ ID NO: 20)
GACCGTAAGGCTTGATGAA
TQSPC (FAM PROBE):
(SEQ ID NO: 21)
CGAGATTCTCCGCGCTGTAGA

Finally, from 8-12 T₀ plants containing the gene of interest were sampled at V6 for Cry34Ab1, Cry35Ab1 and AAD-1 leaf ELISA assays. Multiple leaf punches were sampled. Leaf Cry34Ab1 (Agdia, Inc., Elkart, Ind.), Cry35Ab1 (Acadia BioScience), and AAD-1 (Agdia, Inc., Elkart, Ind.) ELISA assays were performed as per the manufacturer's instructions. The leaf ELISA assays were expressed as parts per million (ppm, or ng protein per mg total plant protein). Total protein concentrations were determined using a PIERCE 660™ nm Protein Assay kit (Thermo Scientific; Rockford, Ill.) following the supplier's instructions.

### Example 5

### Transgene Expression in Maize

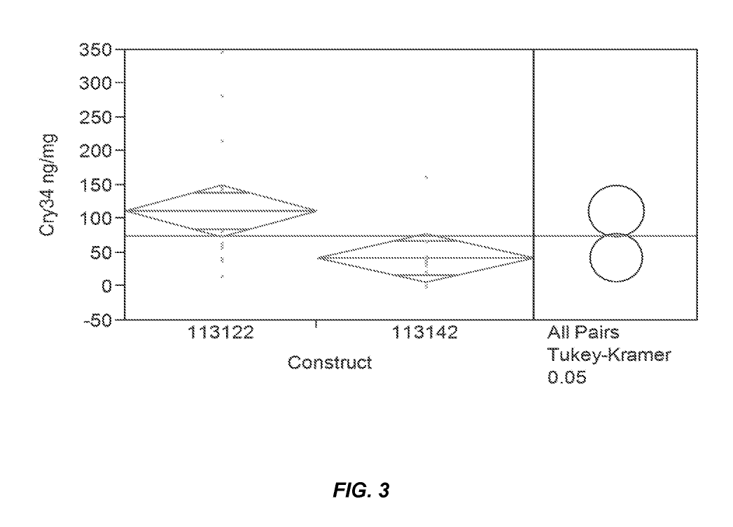

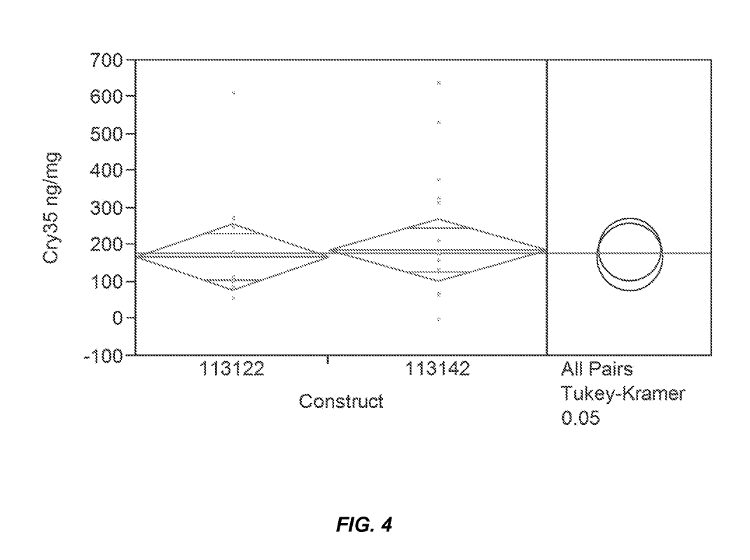

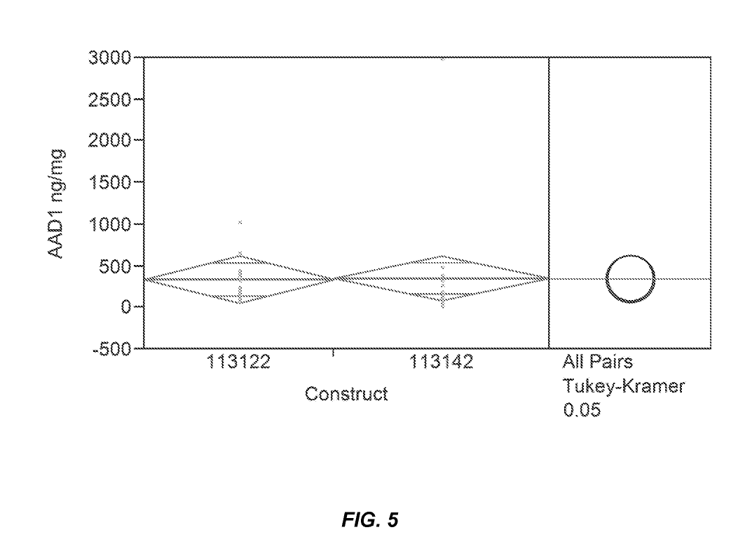

The CRY34 ELISA results indicated that the Rubi3 bidirectional promoter (SEQ ID NO:1) drove expression of Cry34Ab1 and Cry35Ab1 in T₀ events that were transformed with constructs pDAB113122 and pDAB113142. FIGS. 3 and 4 show the results of the analyses for bidirectional promoter from constructs pDA113122 and pDAB113142. The data reveal that there is consistent Cry34 and Cry35 protein production in corn plants using the RUbi3 bidirectional promoter of SEQ ID NO: 1. The expression levels of Cry34 (FIG. 3, Table 1), Cry35 (FIG. 4, Table 2) and AAD1 (FIG. 5, Table 3) were similar in both constructs pDAB113122 and pDAB113142. The data show that Rubi3 bidirectional promoter disclosed in this invention is useful in making transgenic traits for co-expression of two transgenes from a single bi-directional promoter element.

TABLE 1
Mean expression of Cry34 protein from
constructs pDAB113122 and PDAB113142.

| Construct | Mean Expression of Cry34 (ng/mg) |
|---|---|
| pDAB113122 | 112.38 |
| pDAB113142 | 42.87647 |

TABLE 2
Mean expression of Cry35 protein from
constructs pDAB113122 and pDAB113142.

| Construct | Mean Expression of Cry35 (ng/mg) |
|---|---|
| pDAB113122 | 168.8 |
| pDAB113142 | 187.4 |

TABLE 3
Mean expression of Cry35 protein from
constructs pDAB113122 and pDAB113142.

| Construct | Mean expression of AAD1 (ng/mg) |
|---|---|
| pDAB113122 | 339.0 |
| pDAB113142 | 353.4 |

In summary, a novel RUbi3 bidirectional promoter of SEQ ID NO:1 was designed and characterized. Disclosed for the first time is a novel RUbi3 bidirectional promoter of SEQ ID NO: regulatory element for use in gene expression constructs. The Rice Ubiquitin 3 and Zea mays Ubiquitin 1 promoters have been converted into novel synthetic Rice Ubiquitin 3 bi-directional promoter. The novel synthetic Rice Ubiquitin 3 bi-directional promoter comprises a plurality of promoter elements from a Zea mays Ubiquitin-1 promoter and a Rice Ubiquitin 3 promoter that are functional both in both dicots (e.g., soybean) and monocots (e.g., corn). The expression levels of the first and second nucleotides of interest obtained from bi-directional promoter appears to be comparable to uni-directional promoter gene constructs. The bi-directional promoters robustly drive expression of multiple transgene sequences that are fused onto either end of the bi-directional promoter.

While a number of exemplary aspects and embodiments have been discussed above, those of skill in the art will recognize certain modifications, permutations, additions and sub-combinations thereof. It is, therefore, intended that the following appended claims and claims hereafter introduced are interpreted to include all such modifications, permutations, additions and sub-combinations as are within their true spirit and scope.

What is claimed is:

1. A synthetic Rice Ubiquitin-3 bi-directional polynucleotide promoter comprising a plurality of promoter elements from a Zea mays Ubiquitin-1 promoter and a Rice Ubiquitin-3 promoter.

2. The synthetic Rice Ubiquitin-3 bi-directional polynucleotide promoter of claim 1, wherein the promoter elements comprise a minimal core promoter.

3. The synthetic Rice Ubiquitin-3 bi-directional polynucleotide promoter of claim 2, wherein the minimal core promoter comprises a polynucleotide sequence with at least 90% sequence identity to SEQ ID NO:2.

4. The synthetic Rice Ubiquitin-3 bi-directional polynucleotide promoter of claim 1, wherein the promoter elements comprise an intron.

5. The synthetic Rice Ubiquitin-3 bi-directional polynucleotide promoter of claim 4, wherein the intron comprises a polynucleotide sequence with at least 90% sequence identity to SEQ ID NO:4.

6. The synthetic Rice Ubiquitin-3 bi-directional polynucleotide promoter of claim 4, wherein the intron comprises a polynucleotide sequence with at least 90% sequence identity to SEQ ID NO:6.

7. The synthetic Rice Ubiquitin-3 bi-directional polynucleotide promoter of claim 1, wherein the promoter elements comprise a 5′-UTR.

8. The synthetic Rice Ubiquitin-3 bi-directional polynucleotide promoter of claim 7, wherein the 5′-UTR comprises a polynucleotide sequence with at least 90% sequence identity to SEQ ID NO:3.

9. The synthetic Rice Ubiquitin-3 bi-directional polynucleotide promoter of claim 7, wherein the 5′-UTR comprises a polynucleotide sequence with at least 90% sequence identity to SEQ ID NO:5.

10. The synthetic Rice Ubiquitin-3 bi-directional polynucleotide promoter of claim 1, wherein the promoter elements comprise an upstream promoter element.

11. The synthetic Rice Ubiquitin-3 bi-directional polynucleotide promoter of claim 10, wherein the upstream promoter element comprises a polynucleotide sequence with at least 90% sequence identity to SEQ ID NO:7.

12. The synthetic Rice Ubiquitin-3 bi-directional polynucleotide promoter of claim 1, wherein the Zea mays Ubiquitin-1 promoter comprises a polynucleotide sequence with at least 90% sequence identity to SEQ ID NO:8.

13. The synthetic Rice Ubiquitin-3 bi-directional polynucleotide promoter of claim 1, wherein the Rice Ubiquitin-3 promoter comprises a polynucleotide sequence with at least 90% sequence identity to SEQ ID NO:9.

14. The synthetic Rice Ubiquitin-3 bi-directional polynucleotide promoter of claim 1, the synthetic bi-directional polynucleotide promoter comprising a polynucleotide sequence with at least 90% sequence identity to SEQ ID NO:1.

15. The synthetic Rice Ubiquitin-3 bi-directional polynucleotide promoter of claim 14, comprising a first polynucleotide sequence of interest operably linked to the 3′ end of the synthetic bi-directional polynucleotide comprising a polynucleotide sequence with at least 90% sequence identity to SEQ ID NO:1.

16. The synthetic Rice Ubiquitin-3 bi-directional polynucleotide promoter of claim 14, comprising a second polynucleotide sequence of interest operably linked to the 5′ end of the synthetic bi-directional polynucleotide comprising a polynucleotide sequence with at least 90% sequence identity to SEQ ID NO:1.

17. The synthetic Rice Ubiquitin-3 bi-directional polynucleotide promoter as in claim 15, wherein the polynucleotide sequence of interest comprises a trait.

18. The synthetic Rice Ubiquitin-3 bi-directional polynucleotide promoter of claim 17, wherein the trait is selected from the group consisting of an insecticidal resistance trait, herbicide tolerance trait, nitrogen use efficiency trait, water use efficiency trait, nutritional quality trait, DNA binding trait, selectable marker trait, and any combination thereof.

19. A method for producing a transgenic plant cell, the method comprising:
a) transforming a plant cell with a gene expression cassette comprising a synthetic Rice Ubiquitin-3 bi-directional polynucleotide promoter operably linked to at least one polynucleotide sequence of interest;
b) isolating the transformed plant cell comprising the gene expression cassette; and,
c) producing a transgenic plant cell comprising the synthetic Rice Ubiquitin-3 bi-directional polynucleotide promoter operably linked to at least one polynucleotide sequence of interest.

20. The method of claim 19, wherein transforming a plant cell is performed with a plant transformation method.

21. The method of claim 20, wherein the plant transformation method is selected from the group consisting of an Agrobacterium-mediated transformation method, a biolistics transformation method, a silicon carbide transformation method, a protoplast transformation method, and a liposome transformation method.

22. The method of claim 19, wherein the nucleotide sequence of interest is constitutively expressed throughout the transgenic plant cell.

23. The method of claim 19, wherein the nucleotide sequence of interest is stably integrated into the genome of the transgenic plant cell.

24. The method of claim 19, the method further comprising the steps of:
e) regenerating the transgenic plant cell into a transgenic plant; and,
f) obtaining the transgenic plant, wherein the transgenic plant comprises the gene expression cassette comprising the synthetic Rice Ubiquitin-3 bi-directional polynucleotide promoter operably linked to at least one polynucleotide sequence of interest.

25. The method of claim 19, wherein the transgenic plant cell is a monocotyledonous transgenic plant cell or a dicotyledonous transgenic plant cell.

26. The method of claim 25, wherein the dicotyledonous transgenic plant cell is selected from the group consisting of an Arabidopsis plant cell, a tobacco plant cell, a soybean plant cell, a canola plant cell, and a cotton plant cell.

27. The method of claim 25, wherein the monocotyledonous transgenic plant cell is selected from the group consisting of a maize plant cell, a rice plant cell, a Brachypodium plant cell, and a wheat plant cell.

28. The synthetic Rice Ubiquitin-3 bi-directional polynucleotide promoter of claim 19, the synthetic Rice Ubiquitin-3 bi-directional polynucleotide comprising SEQ ID NO:1.

29. The synthetic Rice Ubiquitin-3 bi-directional polynucleotide promoter of claim 19, comprising a first polynucleotide sequence of interest operably linked to the 3′ end of the synthetic Rice Ubiquitin-3 bi-directional polynucleotide promoter comprising SEQ ID NO:1.

30. The synthetic Rice Ubiquitin-3 bi-directional polynucleotide promoter of claim 19, comprising a second polynucleotide sequence of interest operably linked to the 5′ end of the synthetic Rice Ubiquitin-3 bi-directional polynucleotide promoter comprising SEQ ID NO:1.

31. A method for expressing a polynucleotide sequence of interest in a plant cell, the method comprising introducing into the plant cell the polynucleotide sequence of interest operably linked to a synthetic Rice Ubiquitin-3 bi-directional polynucleotide promoter.

32. The method of claim 31, wherein the polynucleotide sequence of interest operably linked to the synthetic Rice Ubiquitin-3 bi-directional polynucleotide promoter is introduced into the plant cell by a plant transformation method.

33. The method of claim 32, wherein the plant transformation method is selected from the group consisting of an Agrobacterium-mediated transformation method, a biolistics transformation method, a silicon carbide transformation method, a protoplast transformation method, and a liposome transformation method.

34. The method of claim 31, wherein the polynucleotide sequence of interest is constitutively expressed throughout the plant cell.

35. The method of claim 31, wherein the polynucleotide sequence of interest is stably integrated into the genome of the plant cell.

36. The method of claim 31, wherein the transgenic plant cell is a monocotyledonous plant cell or a dicotyledonous plant cell.

37. The method of claim 36, wherein the dicotyledonous plant cell is selected from the group consisting of an Arabidopsis plant cell, a tobacco plant cell, a soybean plant cell, a canola plant cell, and a cotton plant cell.

38. The method of claim 36, wherein the monocotyledonous plant cell is selected from the group consisting of a maize plant cell, a rice plant cell, a Brachypodium plant cell, and a wheat plant cell.

39. The synthetic Rice Ubiquitin-3 bi-directional polynucleotide promoter of claim 31, the synthetic Rice Ubiquitin-3 bi-directional polynucleotide comprising at least 90% sequence identity to SEQ ID NO:1.

40. The synthetic Rice Ubiquitin-3 bi-directional polynucleotide promoter of claim 31, comprising a first polynucleotide sequence of interest operably linked to the 3′ end of the synthetic Rice Ubiquitin-3 bi-directional polynucleotide promoter comprising SEQ ID NO:1.

41. The synthetic Rice Ubiquitin-3 bi-directional polynucleotide promoter of claim 31, comprising a second polynucleotide sequence of interest operably linked to the 5′ end of the synthetic Rice Ubiquitin-3 bi-directional polynucleotide promoter comprising SEQ ID NO:1.

42. A transgenic plant cell comprising a synthetic Rice Ubiquitin-3 bi-directional polynucleotide promoter.

43. The transgenic plant cell of claim 42, wherein the transgenic plant cell comprises a transgenic event.

44. The transgenic plant cell of claim 43, wherein the transgenic event comprises an agronomic trait.

45. The transgenic plant cell of claim 44, wherein the agronomic trait is selected from the group consisting of an insecticidal resistance trait, herbicide tolerance trait, nitrogen use efficiency trait, water use efficiency trait, nutritional quality trait, DNA binding trait, selectable marker trait, or any combination thereof.

46. The transgenic plant cell of claim 44, wherein the agronomic trait comprises an herbicide tolerant trait.

47. The transgenic plant cell of claim 46, wherein the herbicide tolerant trait comprises an aad-1 coding sequence.

48. The transgenic plant cell of claim 42, wherein the transgenic plant cell produces a commodity product.

49. The transgenic plant cell of claim 48, wherein the commodity product is selected from the group consisting of protein concentrate, protein isolate, grain, meal, flour, oil, or fiber.

50. The transgenic plant cell of claim 42, wherein the transgenic plant cell is selected from the group consisting of a dicotyledonous plant cell or a monocotyledonous plant cell.

51. The transgenic plant cell of claim 50, wherein the monocotyledonous plant cell is a maize plant cell.

52. The transgenic plant cell of claim 42, the synthetic Rice Ubiquitin-3 bi-directional polynucleotide comprising at least 90% sequence identity to SEQ ID NO:1.


SEQUENCE LISTING (23 sequences)

SEQ ID NO: 1 - DNA, 3303 bp, Artificial Sequence
  Synthetic Rice Ubiquitin 3 Bi directional polynucleotide promoter
tgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcgaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatgcatggaccggaacgccgatctagagaaggtagagagggggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccggtgccgtgagaggaggaggaggccgcctgccgtgccggctcacgtctgccgctccgccacgcaatttctggatgccgacagcggagcaagtccaacggtggagcggaactctcgagaggggtccaggatgtgaagaacaggtaaatcacgcagaagaacccatctctgatagcagctatcgattagaacaacgaatccatattgggtccgtgggaaatacttactgcacaggaagggggcgatctgacgaggccccgccaccggcctcgacccgaggccgaggccgacgaagcgccggcgagtacggcgccgcggcggcctctgcccgtgccctctgcgcgtgggagggagaggccgcggtggtgggggcgcgcgcgcgcgcgcgcgcagctggtgcggcggcgcgggggtcagccgccgagccggcggcgacggaggagcagggcggcgtggacgcgaacttccgatcggttggtcagagtgcgcgagttgggcttagccaattaggtctcaacaatctattgggccgtaaaattcatgggccctggtttgtctaggcccaatatcccgttcatttcagcccacaaatatttccccagaggattattaaggcccacacgcagcttatagcagatcaagtacgatgtttcctgatcgttggatcggaaacgtacggtcttgatcaggcatgccgacttcgtcaaagagaggcggcatgacctgacgcggagttggttccgggcaccgtctggatggtcgtaccgggaccggacacgtgtcgcgcctccaactacatggacacgtgtggtgctgccattgggccgtacgcgtggcggtgaccgcaccggatgctgcctcgcaccgccttgcccacgctttatatagagaggttttctctccattaatcgcatagcgagtcgaatcgaccgaaggggagggggagcgaagctttgcgttctctaatcgcctcgtcaaggtaactaatcaatcacctcgtcctaatcctcgaatctctcgtggtgcccgtctaatctcgcgattttgatgctcgtggtggaaagcgtaggaggatcccgtgcgagttagtctcaatctctcagggtttcgtgcgattttagggtgatccacctcttaatcgagttacggtttcgtgcgattttagggtaatcctcttaatctctcattgatttagggtttcgtgagaatcgaggtagggatctgtgttatttatatcgatctaatagatggattggttttgagattgttctgtcagatggggattgtttcgatatattaccctaatgatgtgtcagatggggattgtttcgatatattaccctaatgatgtgtcagatggggattgtttcgatatattaccctaatgatggataataagagtagttcacagttatgttttgatcctgccacatagtttgagttttgtgatcagatttagtttcacttatttgtgcttagttcggatgggattgttctgatattgttccaatagatgaatagctcgttaggttaaaatctttaggttgagttaggcgacacatagtttatttcctctggatttggattggaattgtgttcttagtttttttcccctggatttggattggaattgtgtggagctgggttagagaattacatctgtatcgtgtacacctacttgaactgtagagcttgggttctaaggtcaatttaatctgtattgtatctggctctttgcctagttgaactgtagtgctgatgttgtactgtgtttttttacccgttttatttgctttactcgtgcaaatcaaatctgtcagatgctagaactaggtggctttattctgtgttcttacatagatctgttgtcctgtagttacttatgtcagttttgttattatctgaagatatttttggttgttgcttgttgatgtggtgtgagctgtgagcagcgctcttatgattaatgatgctgtccaattgtagtgtaatatgatgtgattgatatgttcatctattttgagctgacagtaccgatatcgtaggatctggtgccaacttattctccagctgcttttttttacctatgttaattccaatcctttcttgcctcttccag

SEQ ID NO: 2 - DNA, 215 bp, Zea mays
agagggtgtggagggggtgtctatttattacggcgggcgaggaagggaaagcgaaggagcggtgggaaaggaatcccccgtagctgccggtgccgtgagaggaggaggaggccgcctgccgtgccggctcacgtctgccgctccgccacgcaatttctggatgccgacagcggagcaagtccaacggtggagcggaactctcgagaggggtccag

SEQ ID NO: 3 - DNA, 82 bp, Zea mays
cttgaagcggaggtgccgacgggtggatttgggggagatctggttgtgtgtgtgtgcgctccgaacaacacgaggttgggga

SEQ ID NO: 4 - DNA, 1015 bp, Zea mays
tgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcgaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatgcatggaccggaacgccgatctagagaaggtagagagggggggggggggggaggacgagcggcgtac

SEQ ID NO: 5 - DNA, 67 bp, Oryza sativa
atagcgagtcgaatcgaccgaaggggagggggagcgaagctttgcgttctctaatcgcctcgtcaag

SEQ ID NO: 6 - DNA, 1139 bp, Oryza sativa
gtaactaatcaatcacctcgtcctaatcctcgaatctctcgtggtgcccgtctaatctcgcgattttgatgctcgtggtggaaagcgtaggaggatcccgtgcgagttgtctcaatctctcagggtttcgtgcgattttagggtgatccacctcttaatcgagttacggtttcgtgcgattttagggtaatcctcttaatctctcattgatttagggtttcgtgagaatcgaggtagggatctgtgttatttatatcgatctaatagatggattggttttgagattgttctgtcagatggggattgtttcgatatattaccctaatgatgtgtcagatggggattgtttcgatatattaccctaatgatgtgtcagatggggattgtttcgatatattaccctaatgatggataataagagtagttcacagttatgttttgatcctgccacatagtttgagttttgtgatcagatttagtttcacttatttgtgcttagttcggatgggattgttctgatattgttccaatagatgaatagctcgttaggttaaaatctttaggttgagttaggcgacacatagtttatttcctctggatttggattggaattgtgttcttagtttttttcccctggatttggattggaattgtgtggagctgggttagagaattacatctgtatcgtgtacacctacttgaactgtagagcttgggttctaaggtcaatttaatctgtattgtatctggctctttgcctagttgaactgtagtgctgatgttgtactgtgtttttttacccgttttatttgctttactcgtgcaaatcaaatctgtcagatgctagaactaggtggctttattctgtgttcttacatagatctgttgtcctgtagttacttatgtcagttttgttattatctgaagatatttttggttgttgcttgttgatgtggtgtgagctgtgagcagcgctcttatgattaatgatgctgtccaattgtagtgtaatatgatgtgattgatatgttcatctattttgagctgacagtaccgatatcgtaggatctggtgccaacttattctccagctgcttttttttacctatgttaattccaatcctttcttgcctcttccag

SEQ ID NO: 7 - DNA, 783 bp, Oryza sativa
gatgtgaagaacaggtaaatcacgcagaagaacccatctctgatagcagctatcgattagaacaacgaatccatattgggtccgtgggaaatacttactgcacaggaagggggcgatctgacgaggccccgccaccggcctcgacccgaggccgaggccgacgaagcgccggcgagtacggcgccgcggcggcctctgcccgtgccctctgcgcgtgggagggagaggccgcggtggtgggggcgcgcgcgcgcgcgcgcgcagctggtgcggcggcgcgggggtcagccgccgagccggcggcgacggaggagcagggcggcgtggacgcgaacttccgatcggttggtcagagtgcgcgagttgggcttagccaattaggtctcaacaatctattgggccgtaaaattcatgggccctggtttgtctaggcccaatatcccgttcatttcagcccacaaatatttccccagaggattattaaggcccacacgcagcttatagcagatcaagtacgatgtttcctgatcgttggatcggaaacgtacggtcttgatcaggcatgccgacttcgtcaaagagaggcggcatgacctgacgcggagttggttccgggcaccgtctggatggtcgtaccgggaccggacacgtgtcgcgcctccaactacatggacacgtgtggtgctgccattgggccgtacgcgtggcggtgaccgcaccggatgctgcctcgcaccgccttgcccacgctttatatagagaggttttctctccattaatcgc

SEQ ID NO: 8 - DNA, 1313 bp, Zea mays
tgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcgaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatgcatggaccggaacgccgatctagagaaggtagagagggggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccggtgccgtgagaggaggaggaggccgcctgccgtgccggctcacgtctgccgctccgccacgcaatttctggatgccgacagcggagcaagtccaacggtggagcggaactctcgagaggggtccag

SEQ ID NO: 9 - DNA, 1990 bp, Oryza sativa
gatgtgaagaacaggtaaatcacgcagaagaacccatctctgatagcagctatcgattagaacaacgaatccatattgggtccgtgggaaatacttactgcacaggaagggggcgatctgacgaggccccgccaccggcctcgacccgaggccgaggccgacgaagcgccggcgagtacggcgccgcggcggcctctgcccgtgccctctgcgcgtgggagggagaggccgcggtggtgggggcgcgcgcgcgcgcgcgcgcagctggtgcggcggcgcgggggtcagccgccgagccggcggcgacggaggagcagggcggcgtggacgcgaacttccgatcggttggtcagagtgcgcgagttgggcttagccaattaggtctcaacaatctattgggccgtaaaattcatgggccctggtttgtctaggcccaatatcccgttcatttcagcccacaaatatttccccagaggattattaaggcccacacgcagcttatagcagatcaagtacgatgtttcctgatcgttggatcggaaacgtacggtcttgatcaggcatgccgacttcgtcaaagagaggcggcatgacctgacgcggagttggttccgggcaccgtctggatggtcgtaccgggaccggacacgtgtcgcgcctccaactacatggacacgtgtggtgctgccattgggccgtacgcgtggcggtgaccgcaccggatgctgcctcgcaccgccttgcccacgctttatatagagaggttttctctccattaatcgcatagcgagtcgaatcgaccgaaggggagggggagcgaagctttgcgttctctaatcgcctcgtcaaggtaactaatcaatcacctcgtcctaatcctcgaatctctcgtggtgcccgtctaatctcgcgattttgatgctcgtggtggaaagcgtaggaggatcccgtgcgagttagtctcaatctctcagggtttcgtgcgattttagggtgatccacctcttaatcgagttacggtttcgtgcgattttagggtaatcctcttaatctctcattgatttagggtttcgtgagaatcgaggtagggatctgtgttatttatatcgatctaatagatggattggttttgagattgttctgtcagatggggattgtttcgatatattaccctaatgatgtgtcagatggggattgtttcgatatattaccctaatgatgtgtcagatggggattgtttcgatatattaccctaatgatggataataagagtagttcacagttatgttttgatcctgccacatagtttgagttttgtgatcagatttagtttcacttatttgtgcttagttcggatgggattgttctgatattgttccaatagatgaatagctcgttaggttaaaatctttaggttgagttaggcgacacatagtttatttcctctggatttggattggaattgtgttcttagtttttttcccctggatttggattggaattgtgtggagctgggttagagaattacatctgtatcgtgtacacctacttgaactgtagagcttgggttctaaggtcaatttaatctgtattgtatctggctctttgcctagttgaactgtagtgctgatgttgtactgtgtttttttacccgttttatttgctttactcgtgcaaatcaaatctgtcagatgctagaactaggtggctttattctgtgttcttacatagatctgttgtcctgtagttacttatgtcagttttgttattatctgaagatatttttggttgttgcttgttgatgtggtgtgagctgtgagcagcgctcttatgattaatgatgctgtccaattgtagtgtaatatgatgtgattgatatgttcatctattttgagctgacagtaccgatatcgtaggatctggtgccaacttattctccagctgcttttttttacctatgttaattccaatcctttcttgcctcttccag

SEQ ID NO: 10 - DNA, 17 bp, Artificial Sequence
  Forward Primer TQ.8v6.1.F
gccataccctccagttg

SEQ ID NO: 11 - DNA, 23 bp, Artificial Sequence
  Reverse Primer TQ.8v6.1.R
gccgttgatggagtagtagatgg

SEQ ID NO: 12 - DNA, 18 bp, Artificial Sequence
  Probe TQ.8v6.1.MGB.P
ccgaatccaacggcttca

SEQ ID NO: 13 - DNA, 18 bp, Artificial Sequence
  Forward Primer InvertaseF
tggcggacgacgacttgt

SEQ ID NO: 14 - DNA, 19 bp, Artificial Sequence
  Reverse Primer InvertaseR
aaagtttggaggctgccgt

SEQ ID NO: 15 - DNA, 21 bp, Artificial Sequence
  InvertaseProbe
cgagcagaccgccgtgtactt

SEQ ID NO: 16 - DNA, 20 bp, Artificial Sequence
  AAD1 Forward Primer
tgttcggttccctctaccaa

SEQ ID NO: 17 - DNA, 22 bp, Artificial Sequence
  AAD1 Reverse Primer
caacatccatcaccttgactga

SEQ ID NO: 18 - DNA, 24 bp, Artificial Sequence
  AAD1 Probe
cacagaaccgtcgcttcagcaaca

SEQ ID NO: 19 - DNA, 19 bp, Artificial Sequence
  SPC1a Primer
cttagctggataacgccac

SEQ ID NO: 20 - DNA, 19 bp, Artificial Sequence
  SPC1s Primer
gaccgtaaggcttgatgaa

SEQ ID NO: 21 - DNA, 21 bp, Artificial Sequence
  TQSPC (FAM PROBE)
cgagattctccgcgctgtaga

SEQ ID NO: 22 - DNA, 5560 bp, Artificial Sequence
  Gene expression cassette of synthetic bi directional Rice Ubiquitin 3 (Rubi3) promoter driving Cry34Ab1 and Cry35Ab1 transgenes in the plasmid pDAB113122
gcaaaacacacctagactagatttgttttgctaacccaattgatattaattatatatgattaatatttatatgtatatggatttggttaatgaaatgcatctggttcatcaaagaattataaagacacgtgacattcatttaggataagaaatatggatgatctctttctcttttattcagataactagtaattacacataacacacaactttgatgcccacattatagtgattagcatgtcactatgtgtgcatccttttatttcatacattaattaagttggccaatccagaagatggacaagtctaggttgtttaaaggttaccgagctctgactcatgactcatgactcaggacttgtggccgtagcgggaggaggtggtctggatggtgtaggtcacgtgggactggttgccggagccgccctgggtgatgatctcgtactgggacttgttggagtggccgtcgtatttgttggagccggcgaacgggttgtcgaagtagaggctgatctcggcctcgccgttgatggagtagtagatggtgccctcggtgccggtcatgaagccgttggattcggccacgaaggtcttgatctggtcgttggccacgttggtcggggaggtgcgccacctgccgccgtcgagcttggtcttgtcctccagctggagggtgtggccggtcttgttgttcacgtcgatgtgcacctcgcgggcggacatggtgtcgtgtggatccggtacctcaactaactactgtacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcgaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatgcatggaccggaacgccgatctagagaaggtagagagggggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccggtgccgtgagaggaggaggaggccgcctgccgtgccggctcacgtctgccgctccgccacgcaatttctggatgccgacagcggagcaagtccaacggtggagcggaactctcgagaggggtccaggatgtgaagaacaggtaaatcacgcagaagaacccatctctgatagcagctatcgattagaacaacgaatccatattgggtccgtgggaaatacttactgcacaggaagggggcgatctgacgaggccccgccaccggcctcgacccgaggccgaggccgacgaagcgccggcgagtacggcgccgcggcggcctctgcccgtgccctctgcgcgtgggagggagaggccgcggtggtgggggcgcgcgcgcgcgcgcgcgcagctggtgcggcggcgcgggggtcagccgccgagccggcggcgacggaggagcagggcggcgtggacgcgaacttccgatcggttggtcagagtgcgcgagttgggcttagccaattaggtctcaacaatctattgggccgtaaaattcatgggccctggtttgtctaggcccaatatcccgttcatttcagcccacaaatatttccccagaggattattaaggcccacacgcagcttatagcagatcaagtacgatgtttcctgatcgttggatcggaaacgtacggtcttgatcaggcatgccgacttcgtcaaagagaggcggcatgacctgacgcggagttggttccgggcaccgtctggatggtcgtaccgggaccggacacgtgtcgcgcctccaactacatggacacgtgtggtgctgccattgggccgtacgcgtggcggtgaccgcaccggatgctgcctcgcaccgccttgcccacgctttatatagagaggttttctctccattaatcgcatagcgagtcgaatcgaccgaaggggagggggagcgaagctttgcgttctctaatcgcctcgtcaaggtaactaatcaatcacctcgtcctaatcctcgaatctctcgtggtgcccgtctaatctcgcgattttgatgctcgtggtggaaagcgtaggaggatcccgtgcgagttagtctcaatctctcagggtttcgtgcgattttagggtgatccacctcttaatcgagttacggtttcgtgcgattttagggtaatcctcttaatctctcattgatttagggtttcgtgagaatcgaggtagggatctgtgttatttatatcgatctaatagatggattggttttgagattgttctgtcagatggggattgtttcgatatattaccctaatgatgtgtcagatggggattgtttcgatatattaccctaatgatgtgtcagatggggattgtttcgatatattaccctaatgatggataataagagtagttcacagttatgttttgatcctgccacatagtttgagttttgtgatcagatttagtttcacttatttgtgcttagttcggatgggattgttctgatattgttccaatagatgaatagctcgttaggttaaaatctttaggttgagttaggcgacacatagtttatttcctctggatttggattggaattgtgttcttagtttttttcccctggatttggattggaattgtgtggagctgggttagagaattacatctgtatcgtgtacacctacttgaactgtagagcttgggttctaaggtcaatttaatctgtattgtatctggctctttgcctagttgaactgtagtgctgatgttgtactgtgtttttttacccgttttatttgctttactcgtgcaaatcaaatctgtcagatgctagaactaggtggctttattctgtgttcttacatagatctgttgtcctgtagttacttatgtcagttttgttattatctgaagatatttttggttgttgcttgttgatgtggtgtgagctgtgagcagcgctcttatgattaatgatgctgtccaattgtagtgtaatatgatgtgattgatatgttcatctattttgagctgacagtaccgatatcgtaggatctggtgccaacttattctccagctgcttttttttacctatgttaattccaatcctttcttgcctcttccagatccagatatgactagctgacgcggcagccatgctggacaccaacaaagtctacgagatttccaaccacgcgaacggactttacgcagctacatatctttcgctcgatgactcgggtgtgtctctgatgaacaagaatgatgacgacattgatgactacaacctcaagtggtttctcttccccattgatgatgaccagtacatcattacttcttatgctgccaacaactgcaaagtttggaatgtgaacaatgataagatcaatgtcagcacctacagctcgaccaattcgatccagaagtggcagatcaaagccaatggttcaagctacgttatccaatccgacaatggcaaggtcctcacagctggcactggccaagccctcggtctgatcagactgaccgatgagtcctccaacaacccaaaccagcaatggaaccttacttccgtccaaacaatccagctgccacagaagcccatcattgacacaaagctgaaagactatcctaagtactcacctactggcaacattgacaatgggacgtctccccagctgatgggctggacgctggtgccgtgtatcatggtcaatgacccaaacatcgacaagaacactcagatcaagaccactccttactacatcctcaagaagtatcagtactggcagagagcggttggcagcaatgtggcactcagaccccatgagaagaagtcatacacctatgaatggggaaccgaaatcgatcagaaaacgaccatcatcaacacgctcgggttccagatcaacatagactcgggaatgaagtttgacatcccagaggtgggaggtggcacagatgagatcaagacccagttgaatgaggaactgaagattgagtacagccatgagacgaagatcatggagaagtatcaagaacaaagcgagatcgacaacccgacagatcagagcatgaactcaatagggttcttgaccattactagtttggagctttacagatacaatggctcagagataaggatcatgcagattcagacgtccgacaatgacacctacaacgtgacgagctatccgaaccaccagcaagccttgctgtgagtagttagcttaatcacctagagctcgtttaaactgagggcactgaagtcgcttgatgtgctgaattgtttgtgatgttggtggcgtattttgtttaaataagtaagcatggctgtgattttatcatatgatcgatctttggggttttatttaacacattgtaaaatgtgtatctattaataactcaatgtataagatgtgttcattcttcggttgccatagatctgcttatttgacctgtgatgttttgactccaaaaaccaaaatcacaactcaataaactcatggaatatgtccacctgtttcttgaagagttcatctaccattccagttggcatttatcagtgttgcagcggcgctgtgctttgtaacataacaattgttacggcatatatccaa

SEQ ID NO: 23 - DNA, 5548 bp, Artificial Sequence
  Gene expression cassette of synthetic bi directional Rice Ubiquitin 3 (Rubi3) promoter driving Cry34Ab1 and Cry35Ab1 transgenes in the plasmid pDAB1131
ttggatatatgccgtaacaattgttatgttacaaagcacagcgccgctgcaacactgataaatgccaactggaatggtagatgaactcttcaagaaacaggtggacatattccatgagtttattgagttgtgattttggtttttggagtcaaaacatcacaggtcaaataagcagatctatggcaaccgaagaatgaacacatcttatacattgagttattaatagatacacattttacaatgtgttaaataaaaccccaaagatcgatcatatgataaaatcacagccatgcttacttatttaaacaaaatacgccaccaacatcacaaacaattcagcacatcaagcgacttcagtgccctcagtttaaacgagctctaggtgattaagctaactactcacagcaaggcttgctggtggttcggatagctcgtcacgttgtaggtgtcattgtcggacgtctgaatctgcatgatccttatctctgagccattgtatctgtaaagctccaaactagtaatggtcaagaaccctattgagttcatgctctgatctgtcgggttgtcgatctcgctttgttcttgatacttctccatgatcttcgtctcatggctgtactcaatcttcagttcctcattcaactgggtcttgatctcatctgtgccacctcccacctctgggatgtcaaacttcattcccgagtctatgttgatctggaacccgagcgtgttgatgatggtcgttttctgatcgatttcggttccccattcataggtgtatgacttcttctcatggggtctgagtgccacattgctgccaaccgctctctgccagtactgatacttcttgaggatgtagtaaggagtggtcttgatctgagtgttcttgtcgatgtttgggtcattgaccatgatacacggcaccagcgtccagcccatcagctggggagacgtcccattgtcaatgttgccagtaggtgagtacttaggatagtctttcagctttgtgtcaatgatgggcttctgtggcagctggattgtttggacggaagtaaggttccattgctggtttgggttgttggaggactcatcggtcagtctgatcagaccgagggcttggccagtgccagctgtgaggaccttgccattgtcggattggataacgtagcttgaaccattggctttgatctgccacttctggatcgaattggtcgagctgtaggtgctgacattgatcttatcattgttcacattccaaactttgcagttgttggcagcataagaagtaatgatgtactggtcatcatcaatggggaagagaaaccacttgaggttgtagtcatcaatgtcgtcatcattcttgttcatcagagacacacccgagtcatcgagcgaaagatatgtagctgcgtaaagtccgttcgcgtggttggaaatctcgtagactttgttggtgtccagcatggtgtcgtgtcaactaactactgtaccctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcgaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatgcatggaccggaacgccgatctagagaaggtagagagggggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccggtgccgtgagaggaggaggaggccgcctgccgtgccggctcacgtctgccgctccgccacgcaatttctggatgccgacagcggagcaagtccaacggtggagcggaactctcgagaggggtccaggatgtgaagaacaggtaaatcacgcagaagaacccatctctgatagcagctatcgattagaacaacgaatccatattgggtccgtgggaaatacttactgcacaggaagggggcgatctgacgaggccccgccaccggcctcgacccgaggccgaggccgacgaagcgccggcgagtacggcgccgcggcggcctctgcccgtgccctctgcgcgtgggagggagaggccgcggtggtgggggcgcgcgcgcgcgcgcgcgcagctggtgcggcggcgcgggggtcagccgccgagccggcggcgacggaggagcagggcggcgtggacgcgaacttccgatcggttggtcagagtgcgcgagttgggcttagccaattaggtctcaacaatctattgggccgtaaaattcatgggccctggtttgtctaggcccaatatcccgttcatttcagcccacaaatatttccccagaggattattaaggcccacacgcagcttatagcagatcaagtacgatgtttcctgatcgttggatcggaaacgtacggtcttgatcaggcatgccgacttcgtcaaagagaggcggcatgacctgacgcggagttggttccgggcaccgtctggatggtcgtaccgggaccggacacgtgtcgcgcctccaactacatggacacgtgtggtgctgccattgggccgtacgcgtggcggtgaccgcaccggatgctgcctcgcaccgccttgcccacgctttatatagagaggttttctctccattaatcgcatagcgagtcgaatcgaccgaaggggagggggagcgaagctttgcgttctctaatcgcctcgtcaaggtaactaatcaatcacctcgtcctaatcctcgaatctctcgtggtgcccgtctaatctcgcgattttgatgctcgtggtggaaagcgtaggaggatcccgtgcgagttagtctcaatctctcagggtttcgtgcgattttagggtgatccacctcttaatcgagttacggtttcgtgcgattttagggtaatcctcttaatctctcattgatttagggtttcgtgagaatcgaggtagggatctgtgttatttatatcgatctaatagatggattggttttgagattgttctgtcagatggggattgtttcgatatattaccctaatgatgtgtcagatggggattgtttcgatatattaccctaatgatgtgtcagatggggattgtttcgatatattaccctaatgatggataataagagtagttcacagttatgttttgatcctgccacatagtttgagttttgtgatcagatttagtttcacttatttgtgcttagttcggatgggattgttctgatattgttccaatagatgaatagctcgttaggttaaaatctttaggttgagttaggcgacacatagtttatttcctctggatttggattggaattgtgttcttagtttttttcccctggatttggattggaattgtgtggagctgggttagagaattacatctgtatcgtgtacacctacttgaactgtagagcttgggttctaaggtcaatttaatctgtattgtatctggctctttgcctagttgaactgtagtgctgatgttgtactgtgtttttttacccgttttatttgctttactcgtgcaaatcaaatctgtcagatgctagaactaggtggctttattctgtgttcttacatagatctgttgtcctgtagttacttatgtcagttttgttattatctgaagatatttttggttgttgcttgttgatgtggtgtgagctgtgagcagcgctcttatgattaatgatgctgtccaattgtagtgtaatatgatgtgattgatatgttcatctattttgagctgacagtaccgatatcgtaggatctggtgccaacttattctccagctgcttttttttacctatgttaattccaatcctttcttgcctcttccagatccagatatgactagctgacgcggcagccatgtccgcccgcgaggtgcacatcgacgtgaacaacaagaccggccacaccctccagctggaggacaagaccaagctcgacggcggcaggtggcgcacctccccgaccaacgtggccaacgaccagatcaagaccttcgtggccgaatccaacggcttcatgaccggcaccgagggcaccatctactactccatcaacggcgaggccgagatcagcctctacttcgacaacccgttcgccggctccaacaaatacgacggccactccaacaagtcccagtacgagatcatcacccagggcggctccggcaaccagtcccacgtgacctacaccatccagaccacctcctcccgctacggccacaagtcctgagtcatgagtcatgagtcagagctcggtaacctttaaacaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgc